(19) United States
(12) Patent Application Publication
(10) Pub. No.: US 2026/0103485 A1
(43) Pub. Date: Apr. 16, 2026

(54) COMPOUND BASED ON OXIDATIVE STRESS AND ANTI-AMYLOID AGGREGATION MECHANISM, PREPARATION METHOD THEREFOR AND USE THEREOF

(71) Applicant: SHENZHEN RAYTONE PHARMACEUTICAL CO., LTD, Longhua District, Shenzhen, Guangdong (CN)

(72) Inventors: Yanbo LING, Zhejiang (CN); Jialin XING, Zhejiang (CN); Jiamin CUI, Zhejiang (CN); Boyang JING, Zhejiang (CN); Zhenhua ZHANG, Zhejiang (CN); Chao HE, Zhejiang (CN)

(21) Appl. No.: 19/112,294

(22) PCT Filed: Dec. 29, 2022
(86) PCT No.: PCT/CN2022/143489

§ 371 Date: Mar. 17, 2025

(30) Foreign Application Priority Data
Oct. 14, 2022 (CN) 202211261821.1

(51) Int. Cl. C07J 31/00; A61K 31/575; C07J 41/00

(52) U.S. Cl. CPC C07J 31/006; A61K 31/575; C07J 41/0088

(57) ABSTRACT

The present invention relates to a compound based on oxidative stress and anti-amyloid aggregation mechanism, a preparation method therefor, and a use thereof. The compound of the present invention has a structure represented by formula (I) or formula (II), can change biomechanical characteristics of a lens to ameliorate age-related lens component changes as well as presbyopia and cataract caused thereby, and therefore can treat age-related presbyopia and cataract diseases and produce a synergistic effect.

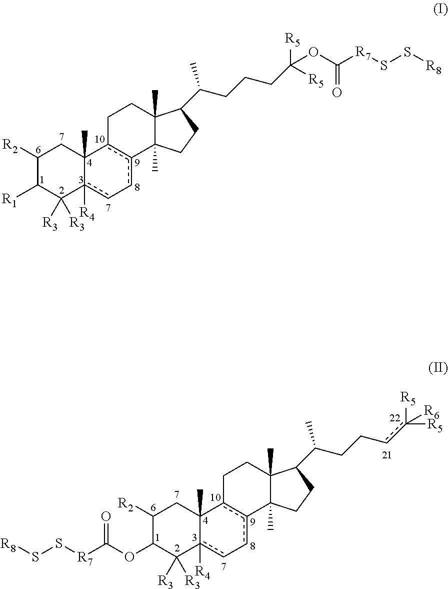

### CROSS-REFERENCE TO RELATED APPLICATIONS

The present application claims the benefit of Chinese Patent Application No. 2022112618211 filed on Oct. 14, 2022, which is hereby incorporated by reference in its entirety.

### TECHNICAL FIELD

The present invention belongs to the field of biomedicine technology, and specifically relates to a compound based on oxidative stress and anti-amyloid aggregation mechanism, and a preparation method and use thereof.

### BACKGROUND ART

The lens of the eye is located in front of the vitreous body and connected to the ciliary body by the circumferential suspensory ligaments of the lens, and is biconvex and flexible. The lens is a biconvex transparent tissue fixed by the suspensory ligaments and suspended behind the iris and in front of the vitreous body. The lens is an important part of the eye's dioptric system and the only refractive medium with accommodation ability. The accommodation ability gradually decreases with age, resulting in presbyopia.

Age-related changes in lens components include changes in oxidative stress, such as an increase in the proportion of protein disulfide bonds, and an increase in amyloid-insoluble proteins (LIAO Xuan, LAN Changjun, Thiol transferase and lens redox regulation [J]. Journal of North Sichuan Medical College, 2011, 26(2): 190-193.; Rich W, Reilly M A. A Review of Lens Biomechanical Contributions to Presbyopia[J]. Current Eye Research, 2022:1-13.). The molecular mechanisms of presbyopia (old eyes) and age-related cataracts are both related to oxidative stress and amyloid-insoluble proteins. (Truscott R J. Presbyopia. Emerging from a blur towards an understanding of the molecular basis for this most common eye condition[J]. Experimental eye research, 2009, 88(2): 241-247.; Rocha K M. Presbyopia on the Horizon[J]. Journal of Refractive Surgery, 2021, 37(S1): S6-S7.; Ho M C, Peng Y J, Chen S J, et al. Senile cataracts and oxidative stress[J]. Journal of clinical gerontology and geriatrics, 2010, 1(1): 17-21.; Zhao L, Chen X J, Zhu J, et al. Lanosterol reverses protein aggregation in cataracts[J]. Nature, 2015, 523 (7562): 607-611.)

So far, the medicines for treating cataracts in clinical practice include: 1. aldose reductase inhibitors, such as pirenoxine (Catalin, Kary Uni, Bernetine), Phacolin, Bendazac Lysine, etc.; 2. antioxidant damage drugs, such as glutathione, taurine, aspirin, etc.; 3. nutritional metabolism drugs, such as vitamins, carotenoids, etc.; 4. Chinese herbal remedies include Shihu Yeguang Wan, Qiju Dihuang Wan, Shijueming San, etc. However, these medicines for treating cataracts have been confirmed by long-term clinical trials to only delay the deterioration of cataracts, but cannot reverse the condition to treat cataracts. So for, no presbyopia treatment drug is marketed, and Vuity approved by the FDA is only a short-acting presbyopia correction medicine.

Meanwhile, the demand for presbyopia and cataract medicines becomes more and more urgent as the number of patients with presbyopia and cataracts is increasing with the growth of the aging population in China. Therefore, clinically, there is a great need for presbyopia and cataract medicines with good efficacy and safety.

### SUMMARY OF THE INVENTION

The present invention provides a compound represented by formula (I), (II), pharmaceutically acceptable salts and isomers thereof:

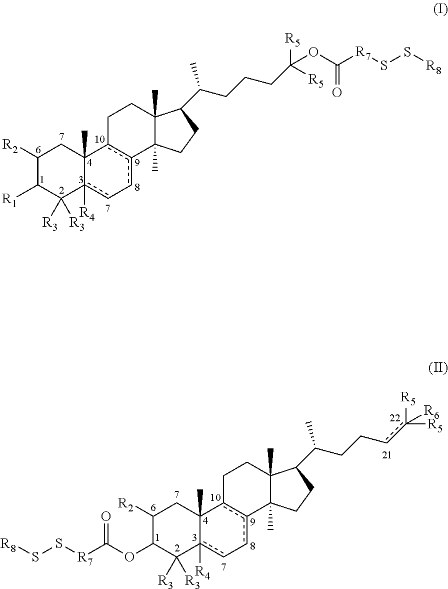

wherein
R₁ is selected from the group consisting of OH, NH₂, and

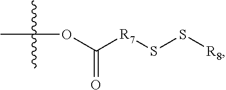

or R₁ is selected from the group consisting of C₁₋₆ alkyl, —OC₁₋₆ alkyl, —OC(═O)C₁₋₆ alkyl, C₁₋₆ heteroalkyl, —OC₁₋₆ heteroalkyl, —OC(═O)C₁₋₆ heteroalkyl, C₃₋₈ cycloalkyl, —OC₃₋₈ cycloalkyl, —OC(═O)C₃₋₈ cycloalkyl, 5-10 membered heterocycloalkyl, C₆₋₁₀ aryl, and 5-10 membered heteroaryl, which are unsubstituted or substituted by 1, 2 or 3 Rs;
R₂ is selected from the group consisting of H, F, Cl, Br, I, NH₂, OH, COOH, and CONH₂;
R₃ is selected from the group consisting of H, F, Cl, Br, I, NH₂, and OH, or R₃ is selected from the group consisting of C₁₋₆ alkyl unsubstituted or substituted by 1, 2 or 3 Rs;
the “ [symbol] ” between carbon atoms No. 3 and 7 is a single or double bond, and when it is a single bond, R₄ is H; and when it is a double bond, R₄ is absent;
the “ [symbol] ” between the adjacent carbon atoms No. 8, 9 and 10 is a single or double bond, and the two bonds cannot be double bonds simultaneously;
R₅ is independently selected from the group consisting of H, and C₁₋₆ alkyl unsubstituted or substituted by 1, 2 or 3 Rs;
the “ [symbol] ” between carbon atoms No. 21 and 22 is a single or double bond, and when it is a single bond, R₆ is selected from the group consisting of OH, NH₂, and

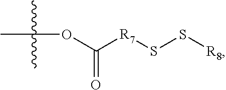

or R₆ is selected from the group consisting of C₁₋₆ alkyl, —OC₁₋₆ alkyl, —OC(═O)C₁₋₆ alkyl, C₁₋₆ heteroalkyl, —OC₁₋₆ heteroalkyl, —OC(═O)C₁₋₆ heteroalkyl, C₃₋₈ cycloalkyl, —OC₃₋₈ cycloalkyl, —OC(═O)C₃₋₈ cycloalkyl, 5-10 membered heterocycloalkyl, C₆₋₁₀ aryl, and 5-10 membered heteroaryl, which are unsubstituted or substituted by 1, 2 or 3 Rs; and when it is a double bond, R₆ is absent;
R₇ is selected from the group consisting of C₁₋₁₂ alkyl and C₁₋₁₂ heteroalkyl, which is unsubstituted or substituted by 1, 2, or 3 Rs;
R₈ is selected from the group consisting of C₁₋₁₂ alkyl and C₁₋₁₂ heteroalkyl, which is unsubstituted or substituted by 1, 2, 3, or 4 Rs;
Rs are independently selected from the group consisting of F, Cl, Br, I, NH₂, OH, COOH, CONH₂, C₁₋₆ alkyl, C₂₋₆ alkenyl, and C₂₋₆ alkynyl;
the “hetero” in the heteroalkyl, heterocycloalkyl, and heteroaryl is independently selected from the following heteroatoms or heteroradicals: —NH—, N, —O—, and —S—; and the number of the heteroatom or heteroradical is independently selected from 1, 2, and 3.

In some embodiments of the present invention, the above Rs are independently selected from the group consisting of F, Cl, Br, I, NH₂, OH, COOH, CONH₂, CH₃, —CH₂CH₃, —(CH₂)₂CH₃, —(CH₂)₃CH₃, —CH═CH₂, and —CH═CH—CH₂, and other variables are as defined herein.

In some embodiments of the present invention, the above R₁ is selected from the group consisting of OH, NH₂, and

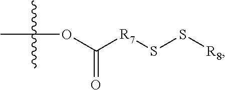

or the above R₁ is selected from the group consisting of

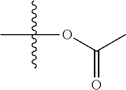 [SMILES: *OC(C)=O]

unsubstituted or substituted by 1, 2, or 3 Rs,
wherein, R₇ is selected from the group consisting of C₁₋₆ alkyl and —NH—C₁₋₆ alkyl-, which are unsubstituted or substituted by 1, 2, or 3 Rs;
R₈ is selected from the group consisting of C₁₋₆ alkyl and —C₁₋₆ alkyl-NH₂, which are unsubstituted or substituted by 1, 2, 3, or 4 Rs; and
other variables are as defined herein.

In some embodiments of the present invention, the above R₁ is selected from the group consisting of OH, NH₂, and

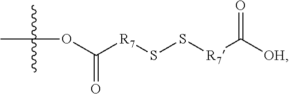

wherein, R₇ and R₇′ are independently selected from the group consisting of C₁₋₆ alkyl unsubstituted or substituted by 1, 2, or 3 Rs, preferably methyl, ethyl and propyl, which are unsubstituted or substituted by 1, 2, or 3 Rs; and
other variables are as defined herein.

In some embodiments of the present invention, the above R₁ is selected from the group consisting of:

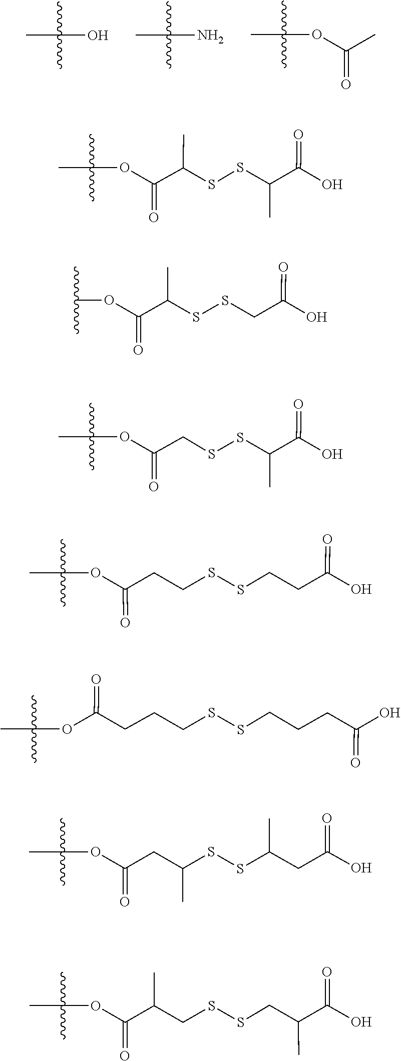

other variables are as defined herein.

In some embodiments of the present invention, the above R₂ is selected from the group consisting of H, F, Cl, NH₂, OH, COOH, and CONH₂, and other variables are as defined herein.

In some embodiments of the present invention, the above R₃ is selected from the group consisting of H, F, Cl, Br, I, NH₂, OH, CH₃, —CH₂OH, —CH₂CH₃, —(CH₂)₂OH, and —(CH₂)₃CH₃, and other variables are as defined herein.

In some embodiments of the present invention, the “ [symbol] ” between carbon atoms No. 3 and 7 is a single bond, and R₄ is H, and other variables are as defined herein.

In some embodiments of the present invention, the above Rs is selected from the group consisting of H, CH₃, —CH₂OH, —CH₂CH₃, and —(CH₂)₂OH, and other variables are as defined herein.

In some embodiments of the present invention, the above R₆ is selected from the group consisting of OH, NH₂, and

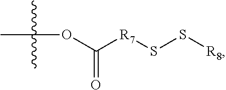

or the above R₆ is

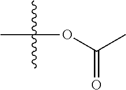 [SMILES: *OC(C)=O]

unsubstituted or substituted by 1, 2, or 3 Rs,
wherein, R₇ is selected from the group consisting of C₁₋₆ alkyl and —NH—C₁₋₆ alkyl-, which are unsubstituted or substituted by 1, 2, or 3 Rs;
R₈ is selected from the group consisting of C₁₋₆ alkyl and —C₁₋₆ alkyl-NH₂, which are unsubstituted or substituted by 1, 2, 3, or 4 Rs;
other variables are as defined herein.

In some embodiments of the present invention, the above R₆ is selected from the group consisting of OH, NH₂, and

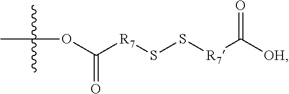

wherein, R₇ and R₇′ are independently selected from the group consisting of C₁₋₆ alkyl unsubstituted or substituted by 1, 2, or 3 Rs, preferably methyl, ethyl and propyl, which are unsubstituted or substituted by 1, 2, or 3 Rs;
other variables are as defined herein.

In some embodiments of the present invention, the above R₆ is selected from the group consisting of:

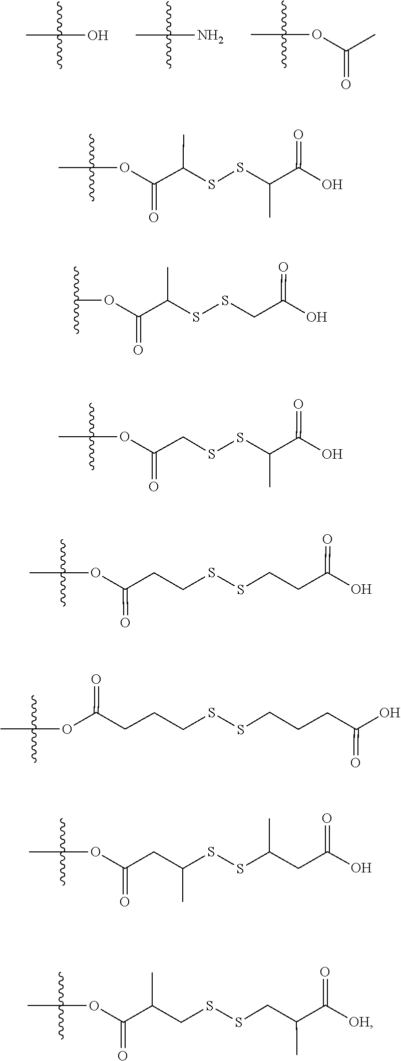

and, other variables are as defined herein.

In some embodiments of the present invention, the R₇ and R₇′ are independently selected from the group consisting of —CH₂—, —(CH₂)₂—, —CH₂CH(CH₃)—, —(CH₂)₃—, —CH₂NH—, and —(CH₂)₂NH—, and other variables are as defined herein.

In some embodiments of the present invention, Rs is selected from the group consisting of —CH₂CH₃, —(CH₂)₂CH₃, —CH₂COOH, —(CH₂)₂COOH, —(CH₂)₃COOH, —CH₂CH(CH₃) COOH, and —CH(CH₃) CH₂COOH, and other variables are as defined herein.

Some other embodiments of the present invention are obtained by any combination of the above variables.

The present invention also provides the following compounds, pharmaceutically acceptable salts and isomers thereof:

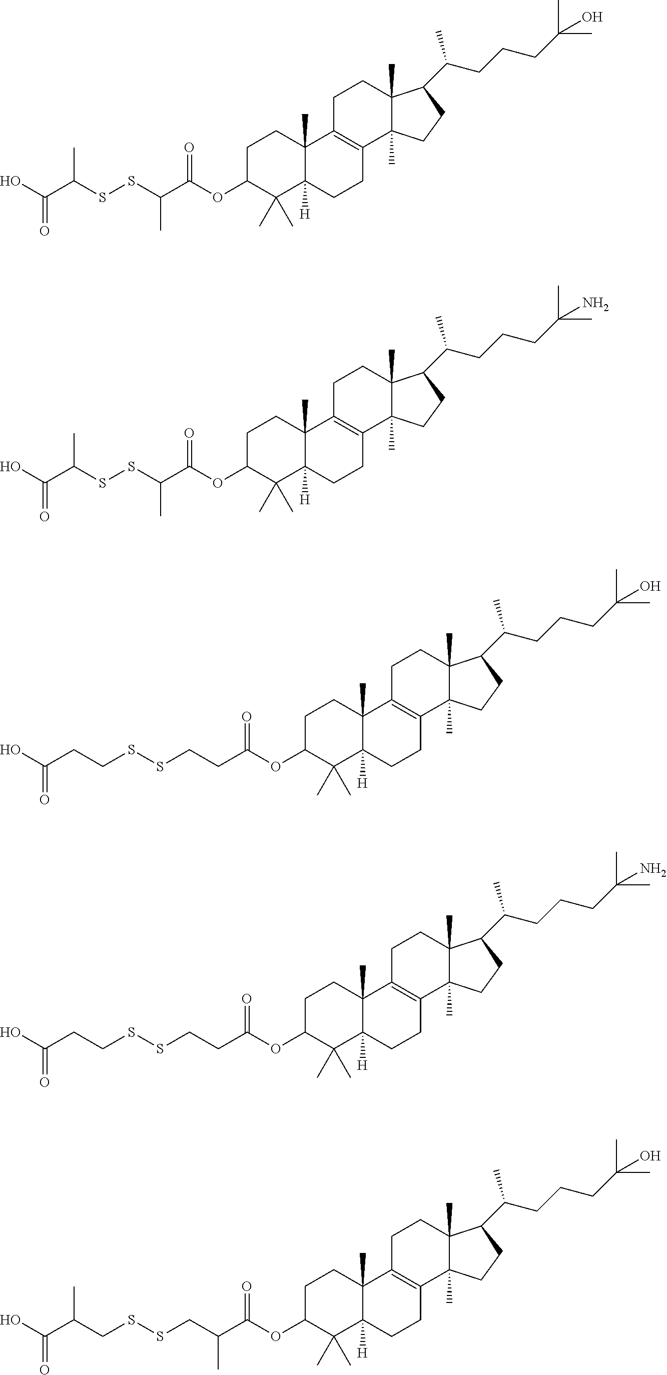

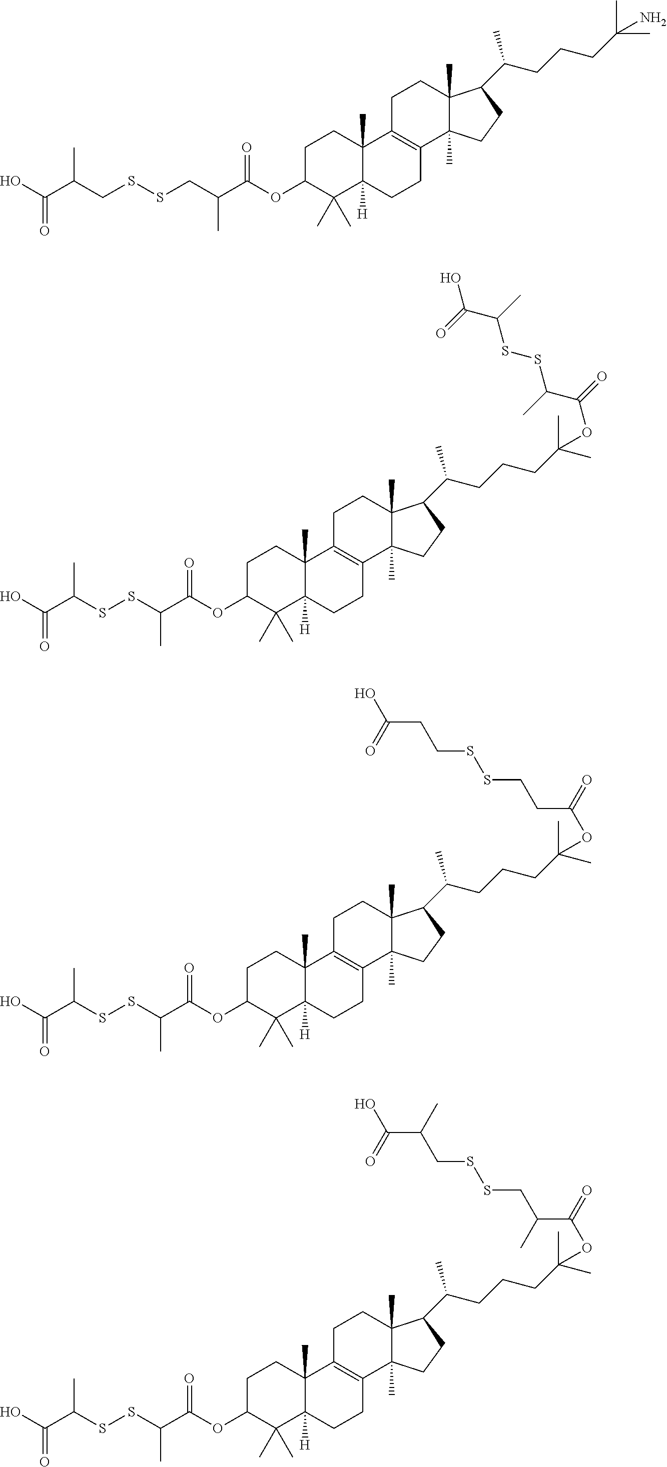 [SMILES: CC(CSSCC(C)C(=O)OC1CC[C@]2(C)C3=C(CC[C@H]2C1(C)C)[C@]1(C)CC[C@H]([C@H](C)CCCC(C)(C)N)[C@@]1(C)CC3)C(=O)O ; CC(SSC(C)C(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3)C(=O)O ; CC(SSC(C)C(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3)C(=O)O ; CC(CSSCC(C)C(=O)OC(C)(C)CCC[C@@H](C)[C@H]1CC[C@@]2(C)C3=C(CC[C@]12C)[C@@]1(C)CCC(OC(=O)C(C)SSC(C)C(=O)O)C(C)(C)[C@@H]1CC3)C(=O)O]

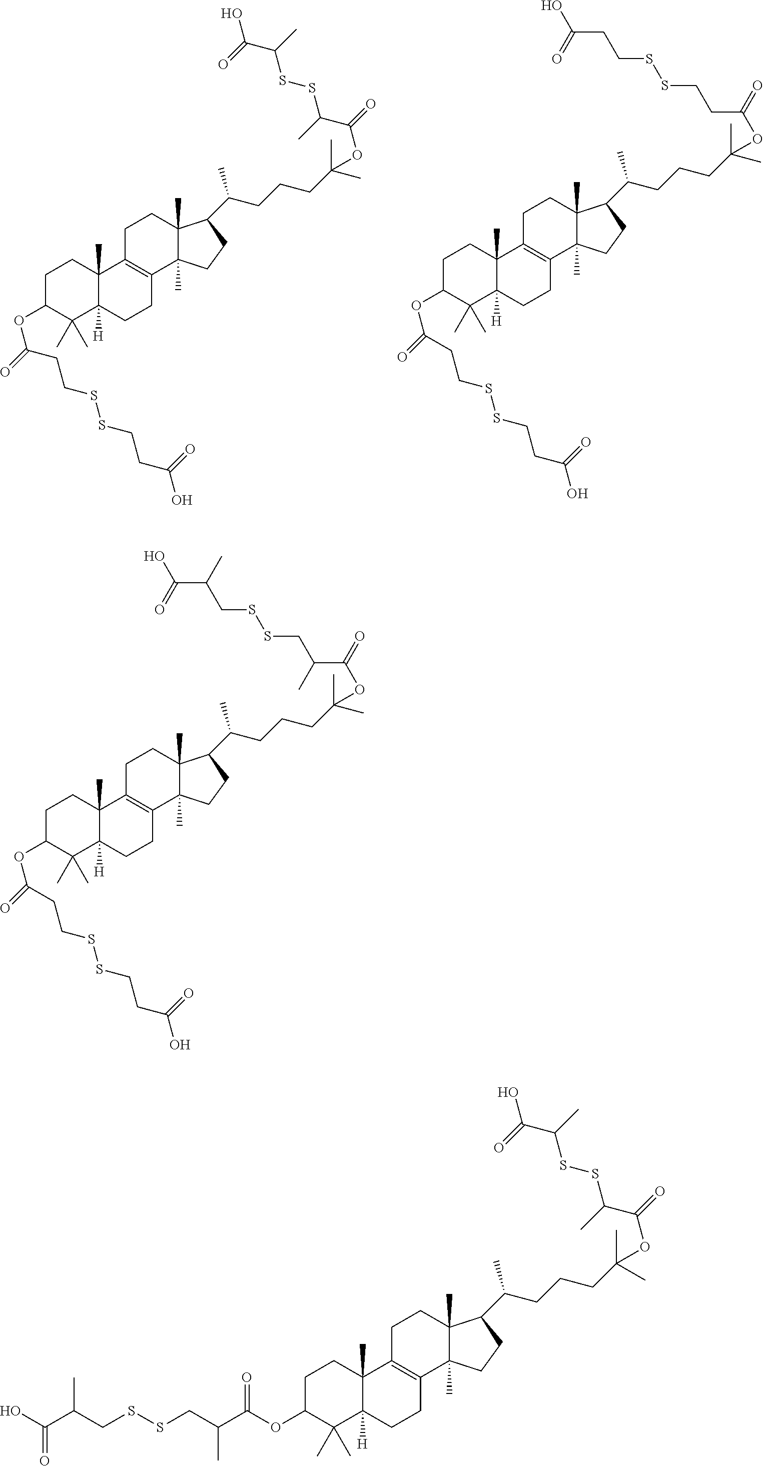 [SMILES: CC(SSC(C)C(=O)OC(C)(C)CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3)C(=O)O ; C[C@H](CCCC(C)(C)OC(=O)CCSSCCC(=O)O)[C@H]1CC[C@@]2(C)C3=C(CC[C@]12C)[C@@]1(C)CCC(OC(=O)CCSSCCC(=O)O)C(C)(C)[C@@H]1CC3 ; CC(CSSCC(C)C(=O)OC(C)(C)CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3)C(=O)O ; CC(CSSCC(C)C(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3)C(=O)O]

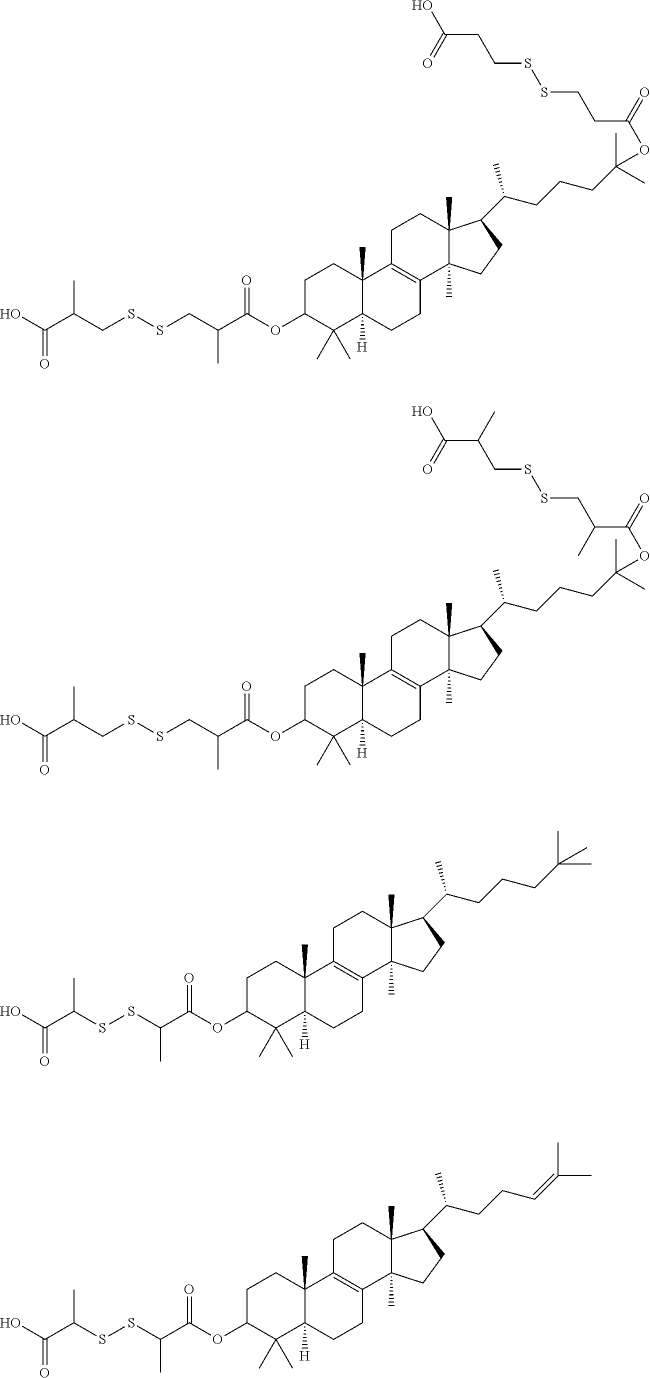 [SMILES: CC(CSSCC(C)C(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3)C(=O)O ; CC(CSSCC(C)C(=O)OC1CC[C@]2(C)C3=C(CC[C@H]2C1(C)C)[C@]1(C)CC[C@H]([C@H](C)CCCC(C)(C)OC(=O)C(C)CSSCC(C)C(=O)O)[C@@]1(C)CC3)C(=O)O ; CC(SSC(C)C(=O)OC1CC[C@]2(C)C3=C(CC[C@H]2C1(C)C)[C@]1(C)CC[C@H]([C@H](C)CCCC(C)(C)C)[C@@]1(C)CC3)C(=O)O ; CC(C)=CCC[C@@H](C)[C@H]1CC[C@@]2(C)C3=C(CC[C@]12C)[C@@]1(C)CCC(OC(=O)C(C)SSC(C)C(=O)O)C(C)(C)[C@@H]1CC3]

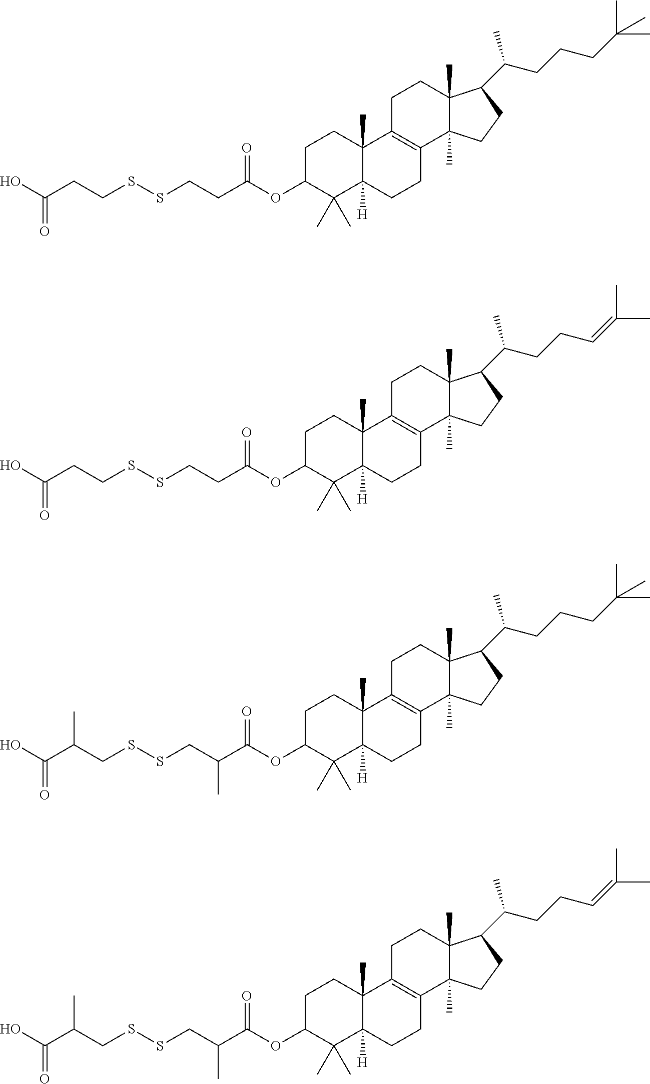 [SMILES: C[C@H](CCCC(C)(C)C)[C@H]1CC[C@@]2(C)C3=C(CC[C@]12C)[C@@]1(C)CCC(OC(=O)CCSSCCC(=O)O)C(C)(C)[C@@H]1CC3 ; CC(C)=CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3 ; CC(CSSCC(C)C(=O)OC1CC[C@]2(C)C3=C(CC[C@H]2C1(C)C)[C@]1(C)CC[C@H]([C@H](C)CCCC(C)(C)C)[C@@]1(C)CC3)C(=O)O ; CC(C)=CCC[C@@H](C)[C@H]1CC[C@@]2(C)C3=C(CC[C@]12C)[C@@]1(C)CCC(OC(=O)C(C)CSSCC(C)C(=O)O)C(C)(C)[C@@H]1CC3]

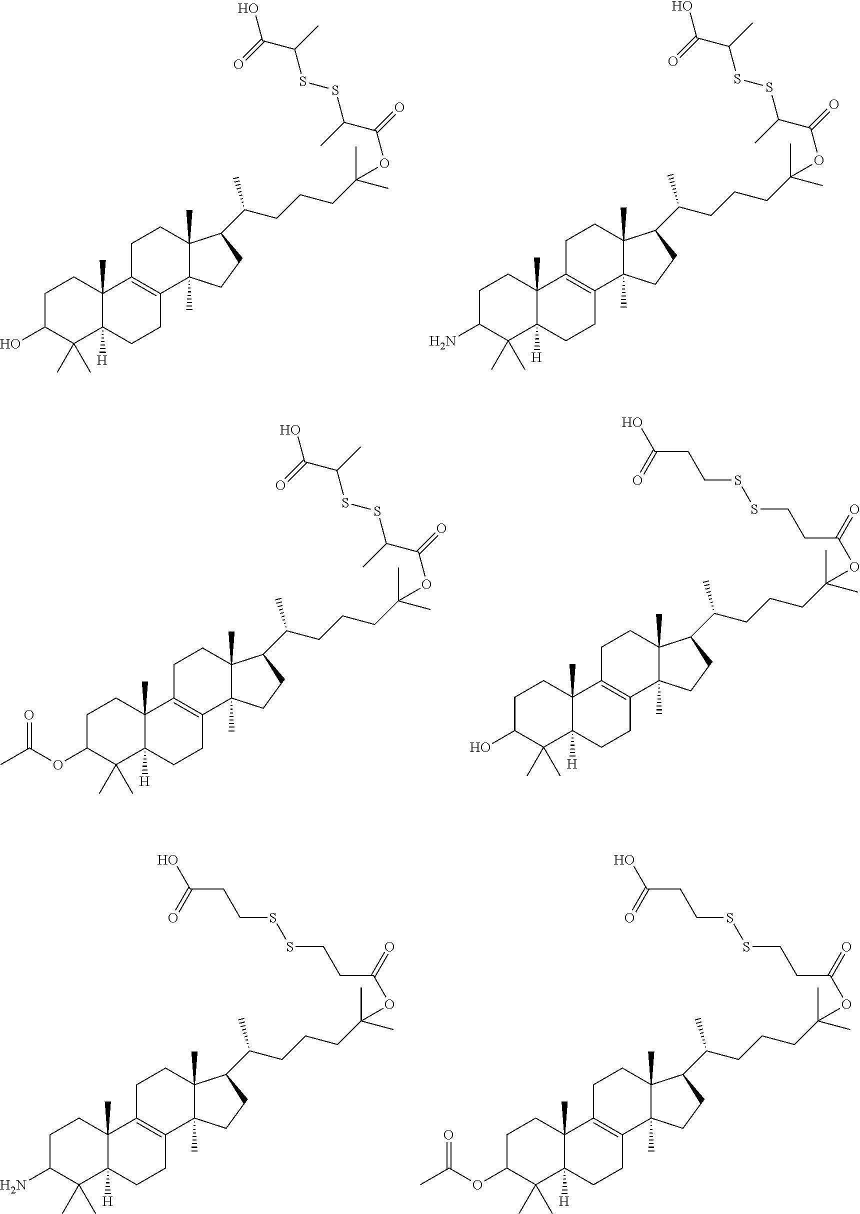

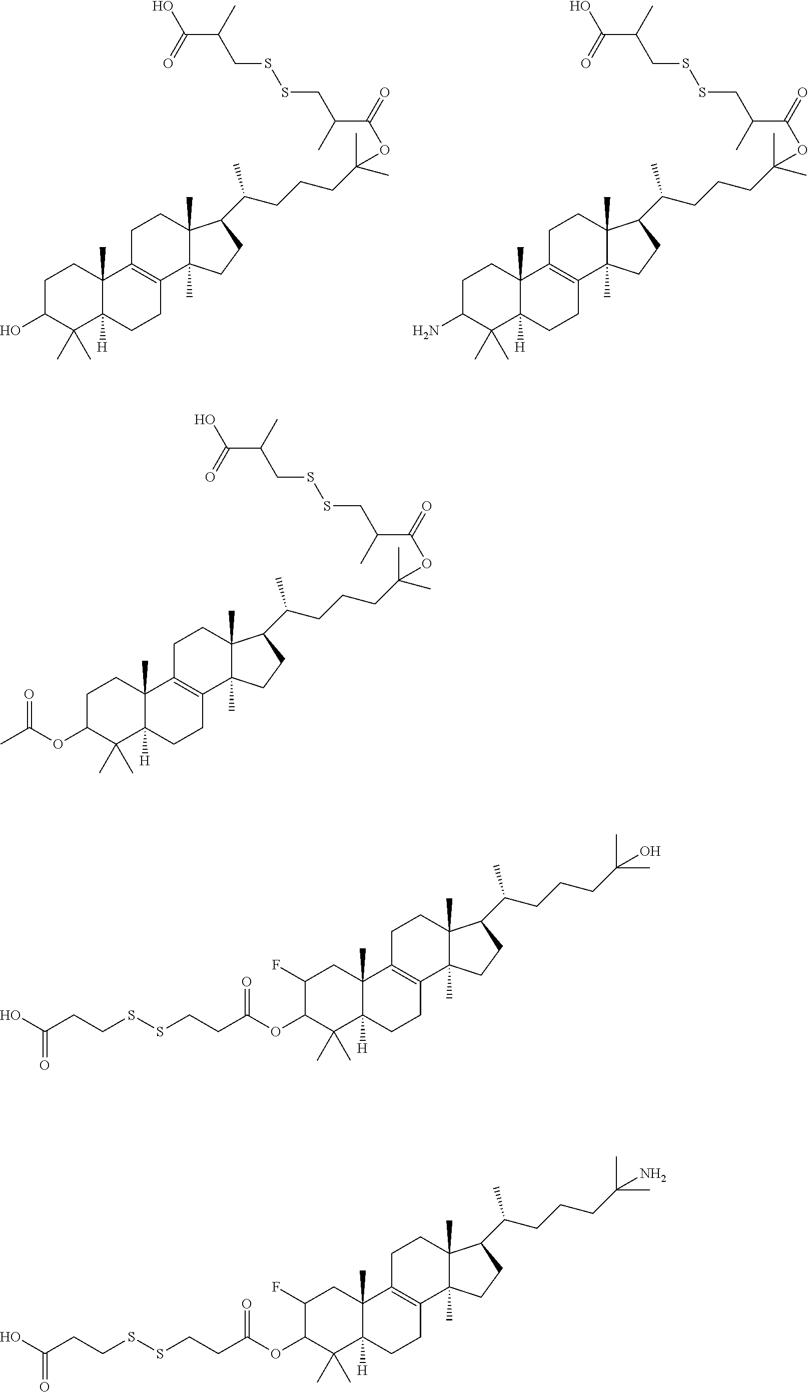

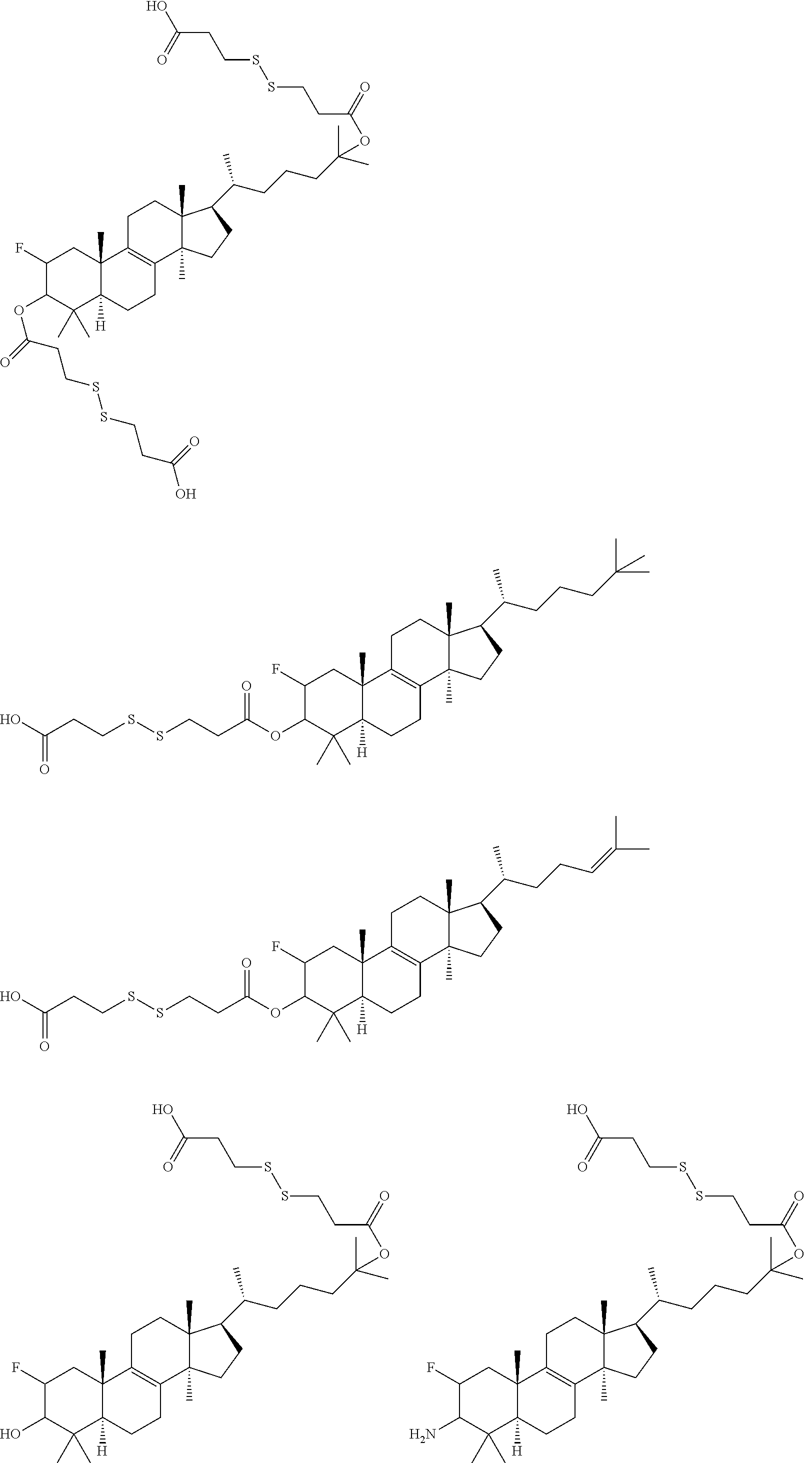

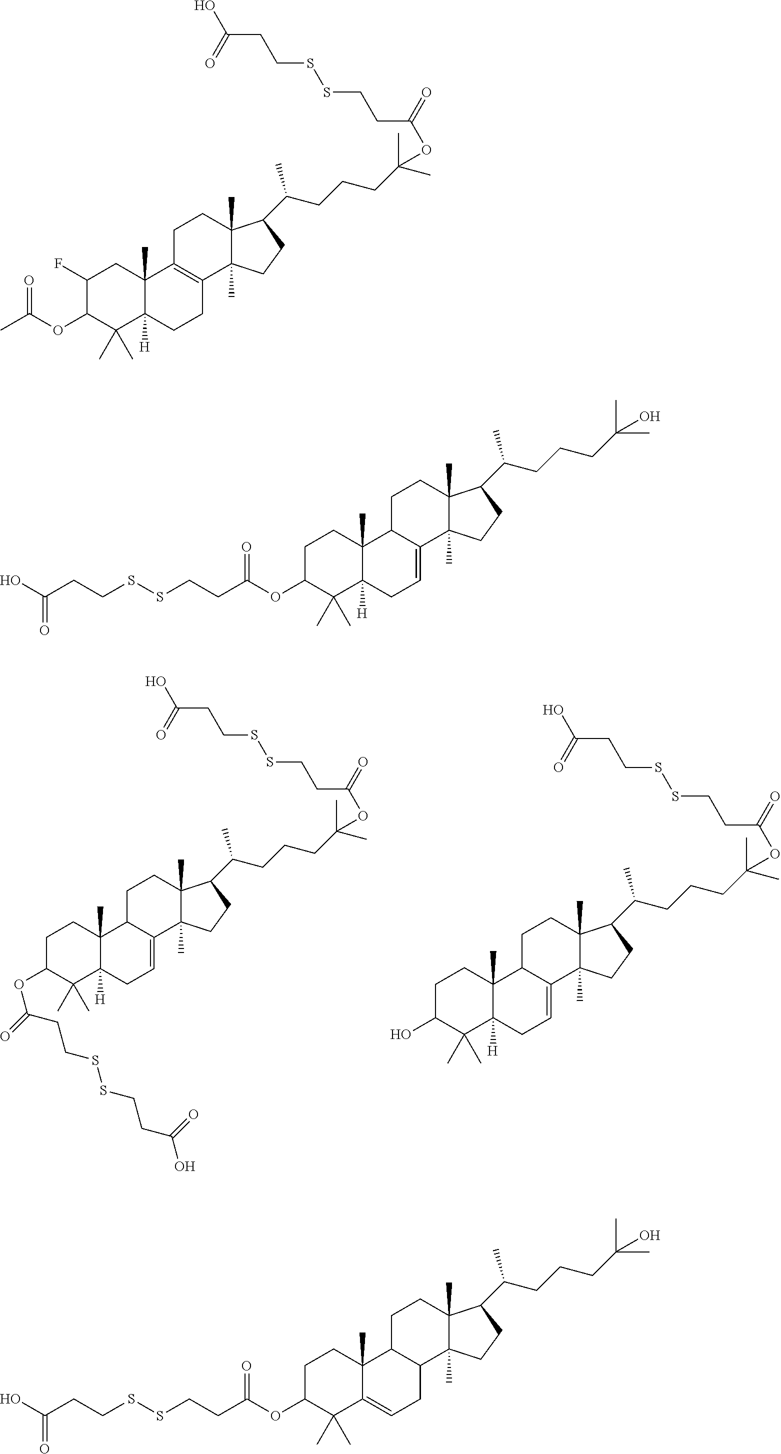

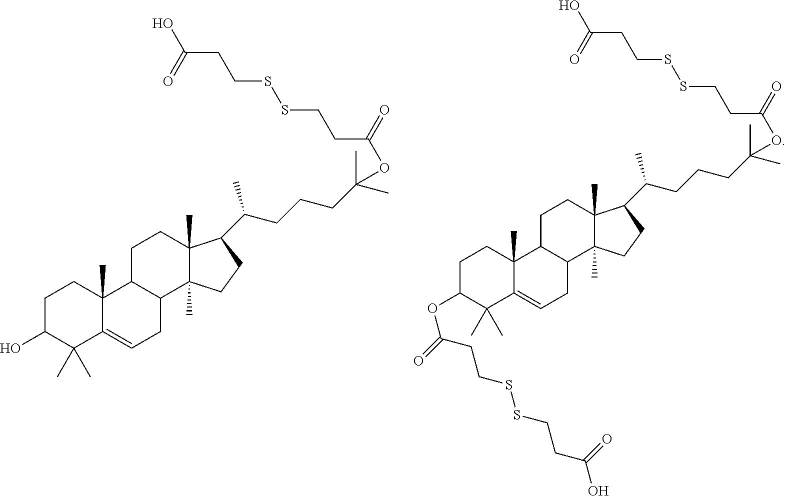 [SMILES: C[C@H](CCCC(C)(C)OC(=O)CCSSCCC(=O)O)[C@H]1CC[C@@]2(C)C3CC=C4C(C)(C)C(O)CC[C@]4(C)C3CC[C@]12C ; C[C@H](CCCC(C)(C)OC(=O)CCSSCCC(=O)O)[C@H]1CC[C@@]2(C)C3CC=C4C(C)(C)C(OC(=O)CCSSCCC(=O)O)CC[C@]4(C)C3CC[C@]12C]

Another aspect of the present invention is to provide a pharmaceutical composition comprising a therapeutically effective amount of the compound, or the pharmaceutically acceptable salt or isomer thereof according to the present invention.

In some embodiments of the present invention, the pharmaceutical composition comprises a therapeutically effective amount of the compound, or the pharmaceutically acceptable salt or isomer thereof according to the present invention as an active ingredient.

In some embodiments of the present invention, the pharmaceutical composition further comprises a pharmaceutically acceptable carrier.

As used herein, the term “pharmaceutically acceptable carrier” includes any or all of a solvent, a dispersion medium, a coating material, a surfactant, an antioxidant, a preservative (e.g., an antibacterial agent, an antifungal agent), an isotonic agent, an absorption delaying agent, a salt, a preservative, a drug stabilizer, a binder, an excipient, a disintegrant, a lubricant, a dye, etc. and a combination thereof, which are well known to those skilled in the art (e.g., see Remington's Pharmaceutical Sciences, 18th Ed. Mack Printing Company, 1990, pp. 1289-1329). Any conventional carrier is contemplated for use in treatment or the pharmaceutical composition except the carriers that are incompatible with the active ingredient.

The dosage form of the pharmaceutical composition of the present invention may be an ophthalmic preparation selected from the group consisting of eye drops, emulsions, gels, eye ointments, sustained-release microspheres, intraocular sustained-release implants, and ocular sustained-release films.

In a preferred embodiment, the ophthalmic preparation is in the form of a solution.

In a preferred embodiment, the ophthalmic preparation is in the form of an emulsion.

Another aspect of the present invention is to provide use of the compound, or the pharmaceutically acceptable salt or isomer thereof according to the present invention, or use of the pharmaceutical composition according to the present invention in preparation of a medicament for treating an ophthalmic disease.

In a preferred embodiment, the medicament for treating an ophthalmic disease is suitable for treating cataracts and/or presbyopia.

In a preferred embodiment, the medicament is a cataract and/or presbyopic ophthalmic preparation.

Preferably, the ophthalmic preparation is selected from the group consisting of eye drops, emulsions, gels, eye ointments, sustained-release microspheres, intraocular sustained-release implants, and ocular sustained-release films.

Further preferably, the ophthalmic preparation is in the form of a solution.

Further preferably, the ophthalmic preparation is in the form of an emulsion.

### Definition and Explanations

Unless otherwise indicated, the following terms and phrases used herein are intended to have the following meanings. A specific term or phrase should not be considered uncertain or unclear in the absence of a special definition, but should be understood in accordance with the ordinary meaning. When a trade name appears herein, it is intended to refer to its corresponding commodity or its active ingredient. The term “pharmaceutically acceptable” used herein means that those compounds, materials, compositions and/or dosage forms, within the scope of reliable medical judgment, are suitable for use in contact with human and animal tissues without excessive toxicity, irritation, allergic reactions or other problems or complications, and are commensurate with a reasonable benefit/risk ratio.

The term “pharmaceutically acceptable salt” refers to a salt of the compound of the present invention, which is prepared from the compound having a specific substituent of the present invention with a relatively nontoxic acid or base. When the compound of the present invention contain a relatively acidic functional group, a base addition salt may be obtained by contacting the neutral form of such compound with a sufficient amount of a base in a pure solution or a suitable inert solvent. Pharmaceutically acceptable base addition salts include sodium, potassium, calcium, ammonium, organic amine or magnesium salts or similar salts. When the compound of the present invention contains a relatively basic functional group, an acid addition salt may be obtained by contacting the neutral form of such compound with a sufficient amount of an acid in a pure solution or a suitable inert solvent. Examples of pharmaceutically acceptable acid addition salts include inorganic acid salts, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, etc.; organic acid salts, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, methanesulfonic acid, etc.; salts of amino acids (such as arginine, etc.); and salts of organic acids such as glucuronic acid. Certain specific compounds of the present invention contain basic and acidic functional groups, and thus can be converted into any base or acid addition salt.

Pharmaceutically acceptable salts of the present invention may be synthesized by conventional chemical methods from parent compounds containing acidic or basic radicals. Generally, the preparation method of such salts is that, in water or an organic solvent or a mixture of both, these compounds in free acid or base form are reacted with a stoichiometric amount of an appropriate base or acid.

In addition to the salt form, the compounds of the present invention may also be provided in a prodrug form. The prodrugs of the compounds described herein are prone to chemical changes under physiological conditions, so as to convert into the compounds of the present invention. In addition, the prodrugs may be converted to the compounds of the present invention by chemical or biochemical methods in vivo.

Certain compounds of the present invention may exist in unsolvated or solvated forms, including hydrated forms. In general, the solvated forms are equivalent to the unsolvated forms, and both are encompassed within the scope of the present invention.

The compounds of the present invention may exist in isomeric forms, including geometric isomers, stereoisomers, enantiomers, diastereomers, tautomers and the like.

The compounds of the present invention may exist in specific geometric or stereoisomeric isomer forms. The present invention contemplates all such compounds, including cis- and trans-isomers, (−)- and (+)-enantiomers, (R)- and (S)-enantiomers, diastereomers, (D)-isomers, (L)-isomers, and racemic mixtures and other mixtures thereof, such as enantiomer- or diastereomer-rich mixtures, all of which are within the scope of the present invention. Additional asymmetric carbon atoms may be present in substituents such as alkyl. All of these isomers and their mixtures are within the scope of the present invention.

Unless otherwise indicated, the term “enantiomer” or “optical isomer” refers to stereoisomers that are mirror images of one another.

Unless otherwise indicated, the term “cis-trans isomers” or “geometric isomers” are from the inability of a ring to rotate freely by double bonds, or by single bonds of ring carbon atoms.

Unless otherwise indicated, the term “diastereomer” refers to stereoisomers that have two or more chiral centers and that are not mirror images of each other.

Unless otherwise indicated, “(D)” or “(+)” indicates dextrorotatory, “(L)” or “(−)” indicates levorotatory, and “(DL)” or “(±)” indicates racemic.

Unless otherwise indicated, the absolute configuration of a stereocenter is indicated by a solid wedge-shaped bond ( [symbol] ) and a dashed wedge-shaped bond ( [symbol] ), the relative configuration of a stereocenter is indicated by a solid straight bond ( [symbol] ) and a dashed straight bond ( [symbol] ), and a solid wedge-shaped bond or a dashed wedge-shaped bond are indicated by a wavy line ( [symbol] ), or a straight solid straight bond and a dashed straight bond are indicated by a wavy line.

Unless otherwise indicated, a chemical bond marked with a wavy line indicates the site attached to an other group.

Unless otherwise stated, for a diyl represented by two chemical bonds, such as —CH₂CH(CH₃)—, —CH₂NH—, etc., its connection direction in the compound, group or structural fragment is arbitrary without limiting to the direction currently shown. The compounds of the present invention may exist in specific tautomers. Unless otherwise specified, the term “tautomer” or “tautomeric form” means that at room temperature, different functional group isomers are in dynamic equilibrium and can quickly convert to each other. If tautomerism is possible (e.g., in solution), the chemical equilibrium of tautomerism can be achieved. For example, proton tautomer (also known as prototropic tautomer) includes interconversion carried out by proton migration, such as ketone-alkenol isomerization and imine-alkenamine isomerization. Valence tautomer includes interconversion carried out by reorganization of some bonding electrons. A specific example of ketone-alkenol tautomerization is the interconversion between two tautomers of pentan-2,4-dione and 4-hydroxypent-3-en-2-one.

Unless otherwise indicated, the terms “one isomer-rich”, “isomer-rich”, “one enantiomer-rich” or “enantiomer-rich” mean that the content of the isomer or enantiomer is less than or equal to 100%, and greater than or equal to 60%, or greater than or equal to 70%, or greater than or equal to 80%, or greater than or equal to 90%, or greater than or equal to 95%, or greater than or equal to 96%, or greater than or equal to 97%, or greater than or equal to 98%, or greater than or equal to 99%, or greater than or equal to 99.5%, or greater than or equal to 99.6%, or greater than or equal to 99.7%, or greater than or equal to 99.8%, or greater than or equal to 99.9%.

Unless otherwise indicated, the term “isomer excess” or “enantiomeric excess” refers to the difference between the relative percentages of two isomers or two enantiomers. For example, if the content of one isomer or enantiomer is 90% and the content of the other isomer or enantiomer is 10%, the isomer or enantiomeric excess (ee value) is 80%.

Optically active (R)- and (S)-isomers as well as D and L isomers may be prepared by chiral synthesis or chiral reagents or other conventional techniques. If one enantiomer of a compound of the present invention is desired, it may be prepared by asymmetric synthesis or derivatization with a chiral auxiliary, wherein the resulting diastereomeric mixture is separated and the auxiliary group is cleaved to provide the pure desired enantiomer. Alternatively, when the molecule contains a basic functional group (such as an amino group) or an acidic functional group (such as a carboxyl group), a diastereomeric salt is formed with an appropriate optically active acid or base, and then the diastereoisomers are separated by conventional methods known in the art, and then the pure enantiomer is recovered. In addition, the separation of enantiomers and diastereomers is usually accomplished by using chromatography, which uses a chiral stationary phase and is optionally combined with a chemical derivatization method (for example, carbamate is generated from an amine). The compounds of the present invention may contain non-natural proportions of atomic isotopes on one or more atoms constituting the compound. For example, the compounds may be labeled with radioactive isotopes, such as tritium (³H), iodine-125 (¹²⁵I) or C-14 (¹⁴C). For another example, hydrogen may be substituted with deuterium to form deuterated drugs. The bond between deuterium and carbon is stronger than the bond between ordinary hydrogen and carbon. Compared with undeuterated drugs, deuterated drugs have the advantages of reducing toxic and side effects, increasing drug stability, enhancing therapeutic effects, and extending the biological half-life of drugs. All isotopic composition changes of the compounds of the present invention, whether radioactive or not, are included in the scope of the present invention. The term “pharmaceutically acceptable carrier” refers to any preparation or carrier medium that can deliver an effective amount of the active substance of the present invention, does not interfere with the biological activity of the active substance, and has no toxic side effects on the host or patient. Representative carriers include water, oil, vegetables and minerals, cream bases, lotion bases, ointment bases, etc. These bases include suspending agents, viscosity enhancers, transdermal enhancers, etc. Their preparations are well known to the person skilled in the field of cosmetics or topical drugs.

“Optional” or “optionally” means that the subsequently described event or circumstance may, but is not necessary to occur, and that the description includes instances where said event or circumstance occurs and instances where the event or circumstance does not occur.

The term “substituted” means that any one or more hydrogen atoms on a specific atom are replaced by a substituent, which may include deuterium and variants of hydrogen, provided that the valency of the particular atom is normal and the substituted compound is stable. When the substituent is oxygen (i.e., ═O), it means that two hydrogen atoms are replaced. Oxygen substitution does not occur on aromatic groups. The term “optionally substituted” means that it may or may not be substituted, and unless otherwise specified, the type and number of substituents may be arbitrary on the basis that they are chemical achievable.

When any variable (e.g., R) appears more than once in composition or structure of a compound, its definition at each occurrence is independent. Thus, for example, if a group is substituted with 0 to 2 Rs, the group may be optionally substituted with up to two Rs, and R in each case has an independent choice. In addition, combinations of substituents and/or variants thereof are permissible only if such combinations result in stable compounds.

When the number of a linking group is 0, such as —(CRR)₀—, it means that the linking group is a single bond.

When one of the variables is selected from a single bond, it means that the two groups connected thereto are directly connected. For example, when L in A-L-Z represents a single bond, it means that the structure is actually A-Z.

When a substituent is vacant, it means that the substituent is absent. For example, when X in A-X is vacant, it means that the structure is actually A. When a substituent may be attached to one or more atoms on a ring, the substituent may be bonded to any atom on the ring.

Unless otherwise specified, the term “hetero” means a heteroatom or a heteroradical (i.e., an atom group containing a heteroatom), including atoms other than carbon (C) and hydrogen (H), as well as atom groups containing such heteroatoms, including, for example, oxygen (O), nitrogen (N), sulfur(S), silicon (Si), germanium (Ge), aluminum (Al), boron (B), —O—, —S—, ═O, ═S, —C(═O)O—, —C(═O)—, —C(═S)—, —S(═O), —S(═O)₂—, and optionally substituted —C(═O)N(H)—, —N(H)—, —C(═NH)—, —S(═O)₂N(H)— or —S(═O)N(H)—.

Unless otherwise specified, “ring” means substituted or unsubstituted cycloalkyl, heterocycloalkyl, cycloalkenyl, heterocycloalkenyl, cycloalkynyl, heterocycloalkynyl, aryl or heteroaryl. The so-called ring includes a single ring, a linked ring, a spiro ring, a fused ring or a bridged ring. The number of atoms on a ring is usually defined as the member number of the ring. For example, “5- to 7-membered ring” means a ring in which 5 to 7 atoms arranged. Unless otherwise specified, the ring optionally contains 1 to 3 heteroatoms. Therefore, the “5- to 7-membered ring” includes, for example, phenyl, pyridyl and piperidinyl: on the other hand, the term “5- to 7-membered heterocycloalkyl ring” includes pyridyl and piperidinyl, but excludes phenyl. The term “ring” also includes a ring system containing at least one ring, each ring of which independently meets the above definition. Unless otherwise specified, the term “heterocycle” or “heterocyclyl” means a stable monocyclic, bicyclic or tricyclic ring containing heteroatom(s) or heteroradical(s), which may be saturated, partially unsaturated or unsaturated (aromatic), and which contains carbon atoms and 1, 2, 3 or 4 heteroatoms independently selected from N, O and S in the ring, wherein any of the above heterocycles may be fused to a benzene ring to form a bicyclic ring. The nitrogen and sulfur heteroatoms may be optionally oxidized (i.e., NO and S(O)ₚ, p is 1 or 2). The nitrogen atom may be substituted or unsubstituted (i.e., N or NR, where R is H or other substituents defined herein). The heterocycle may be attached to the pendant group of any heteroatom or carbon atom to form a stable structure. The heterocycle described herein may be substituted at the carbon or nitrogen position if the resulting compound is stable. The nitrogen atom in the heterocycle is optionally quaternized. In a preferred embodiment, when the total number of S and O atoms in the heterocycle exceeds 1, these heteroatoms are not adjacent to each other. In another preferred embodiment, the total number of S and O atoms in the heterocycle does not exceed 1. As used herein, the term “aromatic heterocyclic group” or “heteroaryl” means a stable 5-, 6-, 7-membered monocyclic or bicyclic ring or 7-, 8-, 9- or 10-membered bicyclic heterocyclic aromatic ring, which contains carbon atoms and 1, 2, 3 or 4 heteroatoms independently selected from N, O and S in the ring. The nitrogen atom may be substituted or unsubstituted (i.e., N or NR, where R is H or other substituents defined herein). The nitrogen and sulfur heteroatoms may be optionally oxidized (i.e., NO and S(O)ₚ, where p is 1 or 2). Preferably, the total number of S and O atoms in the aromatic heterocycle does not exceed 1. Bridged rings are also included in the definition of heterocycles. A bridged ring is formed when one or more atoms (i.e., C, O, N or S) connect two non-adjacent carbon atoms or nitrogen atoms. Preferred bridged rings include, but are not limited to, one carbon atom, two carbon atoms, one nitrogen atom, two nitrogen atoms, and a carbon-nitrogen group. It is worth noting that a bridge always converts a monocyclic ring into a tricyclic ring. In a bridged ring, substituents on the ring may also appear on the bridge.

Unless otherwise specified, the term “hydrocarbyl”, “hydrocarbon radical” or its specific concepts (such as alkyl, alkenyl, alkynyl, aryl, etc.) by itself or as part of another substituent means a linear, branched or cyclic hydrocarbon radical or a combination thereof, which may be fully saturated (such as alkyl), mono- or polyunsaturated (such as alkenyl, alkynyl, aryl), may be mono- or poly-substituted, may be monovalent (such as methyl), divalent (such as methylene) or polyvalent (such as methine), may include divalent or polyvalent radical, and may have a specified number of carbon atoms (such as C1-C12 means 1 to 12 carbons, C1-12 is selected from the group consisting of C1, C2, C3, C4, C5, C6, C7, C8, C9, C10, C11 and C12; C3-12 is selected from the group consisting of C3, C4, C5, C6, C7, C8, C9, C10, C11 and C12.). “Hydrocarbyl” includes but is not limited to aliphatic hydrocarbyl and aromatic hydrocarbyl. The aliphatic hydrocarbyl includes chain and ring hydrocarbyl, specifically, it includes, but not limited to, alkyl, alkenyl, alkynyl, cycloalkyl, cycloalkenyl, and cycloalkynyl. The aromatic hydrocarbyl includes, but is not limited to, 6- to 12-membered, preferably 6- to 10-membered aromatic hydrocarbyl. In some examples, the term “hydrocarbyl” means a linear or branched radical or a combination thereof, which may be fully saturated, mono- or poly-unsaturated, and may include divalent and polyvalent radicals. Examples of saturated hydrocarbyls such as alkyl, cycloalkyl, etc. include, but are not limited to, methyl, ethyl, n-propyl, isopropyl, n-butyl, tert-butyl, isobutyl, sec-butyl, isobutyl, cyclohexyl, (cyclohexyl)methyl, cyclopropylmethyl, and homologs or isomers of radicals such as n-pentyl and n-hexyl. Unsaturated hydrocarbyls have one or more double bonds or triple bonds, and examples thereof include, but are not limited to, vinyl, 2-propenyl, ethynyl, 1-propynyl, and 3-propynyl, and higher homologs and isomers. Examples of aromatic hydrocarbyls such as aryl include, but are not limited to, phenyl, naphthyl, tetrahydronaphthyl, indenyl, indanyl, phenanthrenyl, and anthracenyl.

Unless otherwise specified, the term “heterohydrocarbyl” or its specific concepts (such as heteroalkyl, heteroalkenyl, heteroalkynyl, heteroaryl, etc.) by itself or in combination with another term refers to a stable linear, branched or cyclic hydrocarbon radical or combination thereof, consisting of a certain number of carbon atoms and at least one heteroatom. The hydrocarbon radical is defined as described herein. In some examples, the term “heteroalkyl” by itself or in combination with another term refers to a stable linear, branched hydrocarbon radical or combination thereof, consisting of a certain number of carbon atoms and at least one heteroatom. In a typical example, the heteroatom is selected from the group consisting of O, N and S, wherein the nitrogen and sulfur atoms are optionally oxidized and the nitrogen heteroatom is optionally quaternized. The number of the heteroatom or heteroradical may be 1, 2, 3 or 4. The heteroatom or heteroradical may be located at any internal position of the heterohydrocarbyl, including the position where the hydrocarbyl is attached to the rest of the molecule. However, the terms “alkoxy”, “alkylamino” and “alkylthio” (or thioalkoxy) are conventional expressions and refer to those alkyl groups that are connected to the rest of the molecule through an oxygen atom, an amino group or a sulfur atom, respectively.

Unless otherwise specified, the term “cyclohydrocarbyl”, “heterocyclohydrocarbyl” or its specific concepts (such as aryl, heteroaryl, cycloalkyl, heterocycloalkyl, cycloalkenyl, heterocycloalkenyl, cycloalkynyl, heterocycloalkynyl, etc.) by itself or in combination with other terms means a cyclized “hydrocarbyl”, “heterohydrocarbyl”, respectively. In addition, in the case of heterohydrocarbyl or heterocyclohydrocarbyl (such as heteroalkyl, heterocycloalkyl), a heteroatom may occupy the position at which the heterocycle is attached to the rest of the molecule.

Unless otherwise specified, the term “halo” or “halogen” by itself or as a part of another substituent represents a fluorine, chlorine, bromine or iodine atom. In addition, the term “haloalkyl” is intended to include monohaloalkyl and polyhaloalkyl. For example, the term “halo (C₁₋₄) alkyl” is intended to include, but is not limited to, trifluoromethyl, 2,2,2-trifluoroethyl, 4-chlorobutyl, 3-bromopropyl, etc.

### Preparation Method

The present invention provides methods for preparing the disclosed compounds according to traditional organic synthesis methods, as well as matrix synthesis methods or combinatorial synthesis methods. The following schemes describe the proposed synthetic routes. Using these schemes, the following guiding principles and examples, those skilled in the art can develop similar or like methods for preparing the compounds within the scope of the present invention.

It will be appreciated by those skilled in the art that the synthesis of the compounds of the present invention may be accelerated by purchasing the intermediates or protected intermediate compounds described in any of the schemes disclosed herein. It will also be appreciated by those skilled in the art that in the preparation of any of the compounds of the present invention, it is necessary and/or desirable to protect sensitive or reactive groups on any molecule of interest. This may be achieved by means of conventional protecting groups, such as those described in “Protective Groups in Organic Synthesis”, John Wiley & Sons, 1999. These protecting groups may be removed at an appropriate stage using methods known in the art.

The present invention provides a method for preparing the compounds of the present invention, pharmaceutically acceptable salts and isomers thereof, which is selected from at least one of the following schemes:

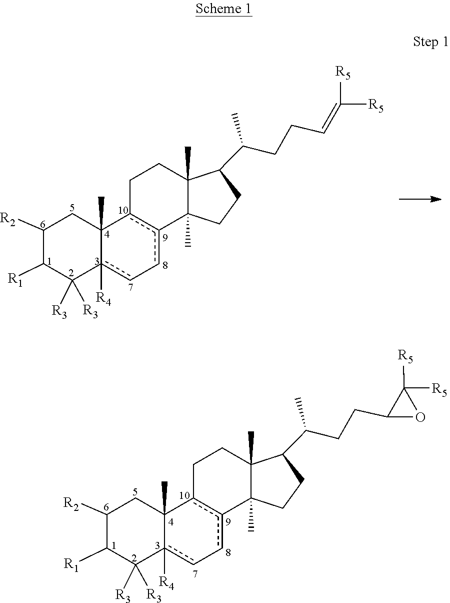

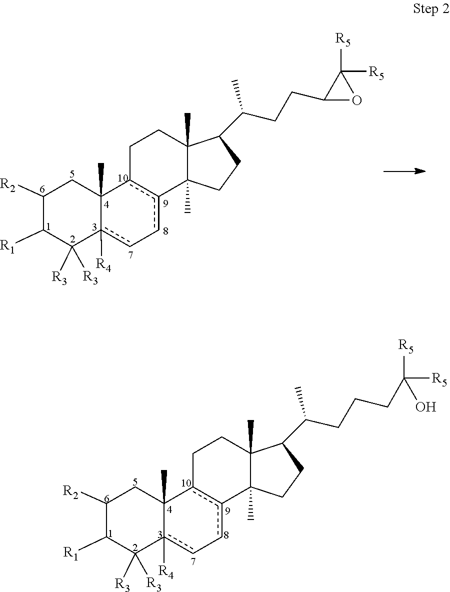

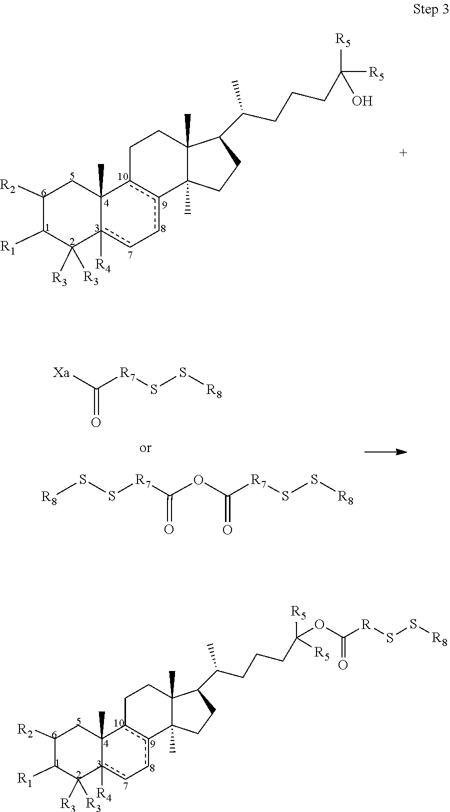

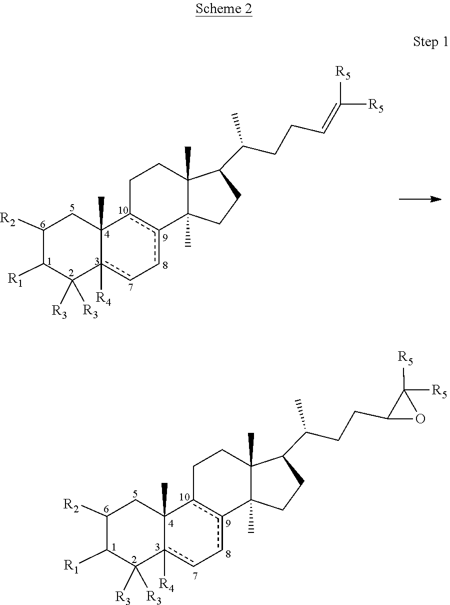

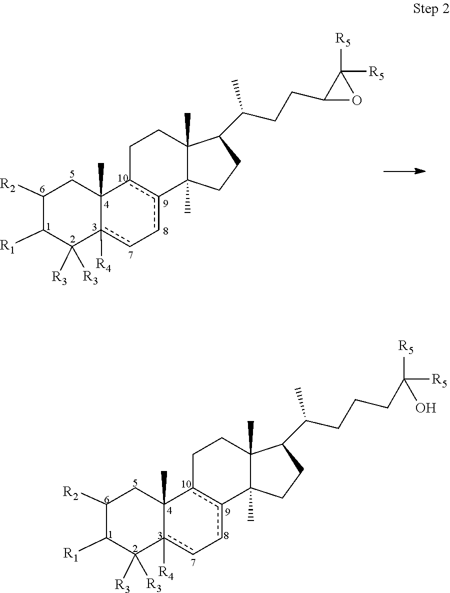

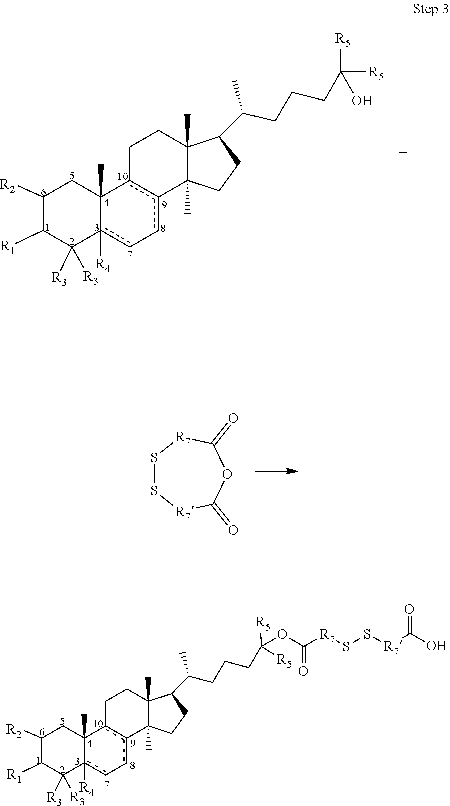

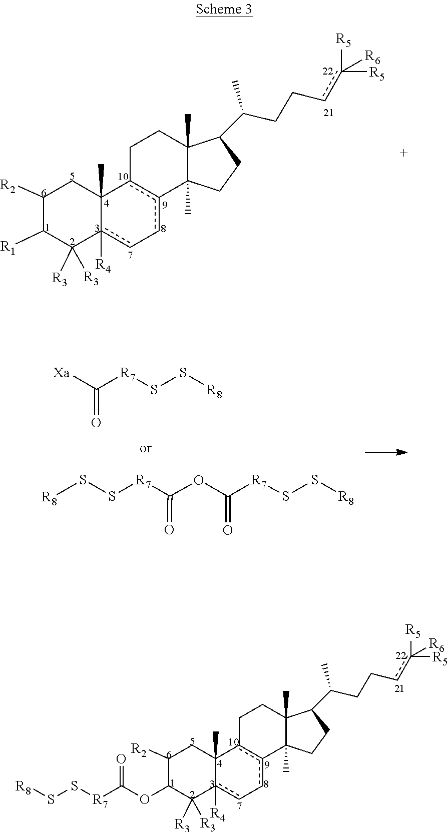

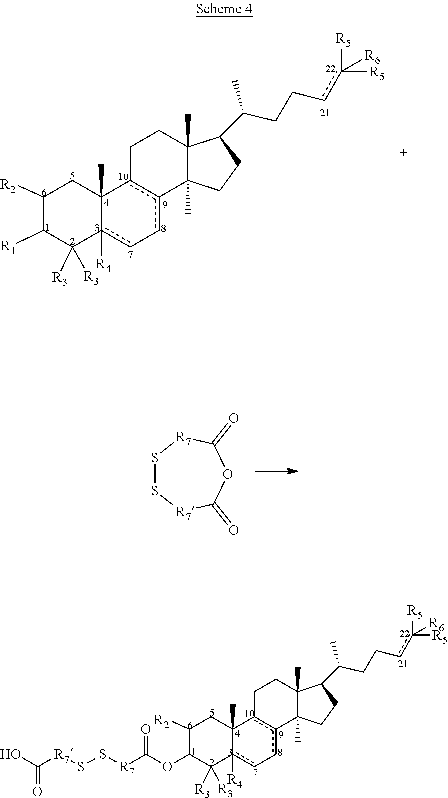

wherein, Xa represents a leaving group, for example, it is selected from the group consisting of hydroxyl, chlorine;
other variables are as defined herein.

### Beneficial Effects

The present invention provides a compound based on oxidative stress and anti-amyloid aggregation mechanism, a preparation method and use thereof. The compound of the present invention improves age-related changes in lens components and the resulting presbyopia and cataract by changing the biomechanical characteristics (anti-oxidative stress and anti-amyloid insoluble protein aggregation) of the lens. On the one hand, the rheology of the lens is changed by reducing the disulfide bonds between lens molecules; and on the other hand, the rheology of the lens is changed by stabilizing the conformation of aggregated proteins to redissolve amyloid proteins. The present invention treats age-related presbyopia and cataract diseases through the above two mechanisms, resulting in a synergistic effect. Therefore, the compound of the present invention can effectively treat presbyopia and/or cataract diseases.

### DETAILED DESCRIPTION

The present invention will be described in detail below by way of examples, but the present invention is not intended to be adversely limited in any way. The present invention has been described in detail herein, and specific embodiments thereof are also disclosed therein. It will be apparent to those skilled in the art that various changes and modifications may be made to the specific embodiments of the present invention without departing from the spirit and scope of the present invention.

The experimental methods in the following examples are all conventional methods unless otherwise specified. If no specific techniques or conditions are specified in the examples, they are carried out according to the techniques or conditions described in the literature in the art or according to the product instructions.

### Example 1

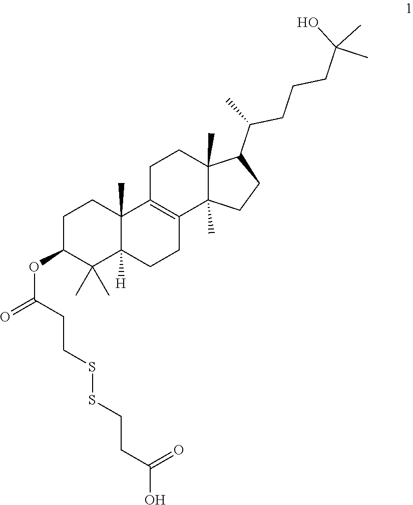

### Synthesis Route:

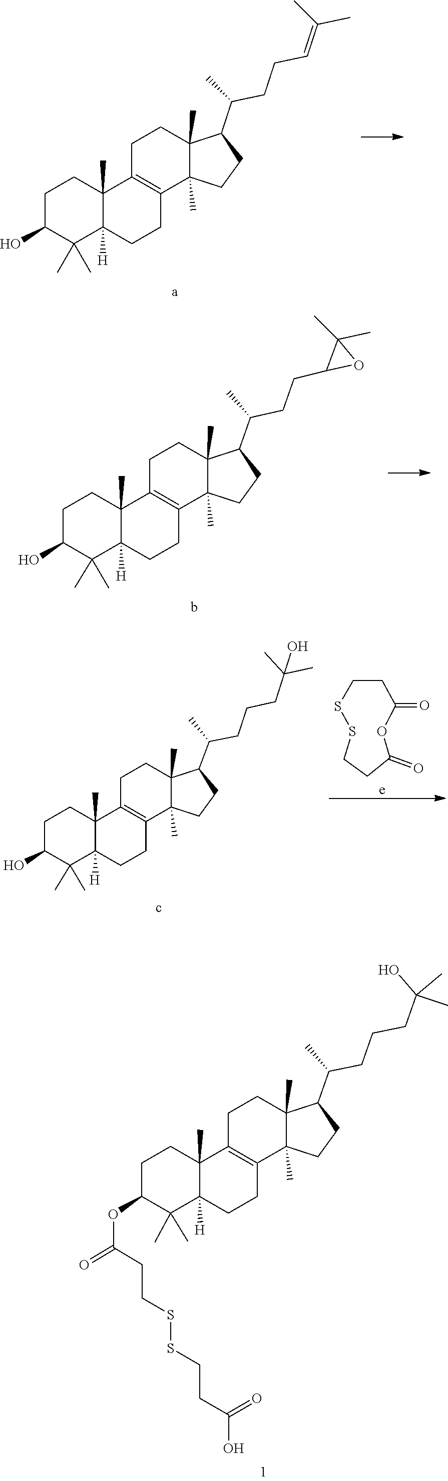

### Step 1: Synthesis of Compound b

Compound a (45.0 g, 52.7 mmol, 50% content) was dissolved in chloroform (450 mL), and m-chloroperoxybenzoic acid (10.7 g, 52.7 mmol, 85% content) was added thereto. The mixture was stirred overnight under nitrogen protection at room temperature. 3 N NaOH (300 mL) was added to the mixture, and the aqueous phase was extracted with dichloromethane (100 mL*3). The combined organic phase was washed with brine (500 mL) and dried over anhydrous sodium sulfate, concentrated in vacuum to obtain the target compound b (25 g, crude product, purity 60.7%) as a white solid.

¹H NMR: (400 MHz, CDCl₃) δ=3.24 (br dd, J=4.3, 11.3 Hz, 1H), 2.69 (t, J=6.1 Hz, 1H), 2.05-1.98 (m, 4H), 1.96-1.90 (m, 1H), 1.75-1.65 (m, 6H), 1.61-1.56 (m, 3H), 1.55-1.33 (m, 9H), 1.31 (s, 3H), 1.27 (s, 3H), 1.00 (s, 3H), 0.98 (s, 3H), 0.92 (d, J=5.9 Hz, 3H), 0.88 (s, 3H), 0.81 (s, 3H), 0.70 (s, 3H);

Mass spectrum: (ESI, positive) m/z 443.5 [M+H]⁺.

### Step 2: Synthesis of Compound c

Compound b (25.0 g, 34.3 mmol, purity 60.7%) was dissolved in tetrahydrofuran (500 mL), lithium aluminum tetrahydride (4.3 g, 113 mmol) was added thereto, and the mixture was stirred overnight in an ice bath with nitrogen protection. An excess of saturated brine was added, and the aqueous phase was extracted with ethyl acetate (100 mL*3). The combined organic phase was washed with brine (500 mL), dried over anhydrous sodium sulfate, concentrated in vacuum to obtain a residue. The residue was triturated with dichloromethane at room temperature for 2 hours to obtain compound c (5.5 g, 36.1% yield) as a white solid.

¹H NMR: (400 MHZ, CDCl₃) δ=3.24 (dd, J=4.5, 11.6 Hz, 1H), 2.08-1.99 (m, 4H), 1.98-1.89 (m, 1H), 1.77-1.65 (m, 5H), 1.64-1.56 (m, 3H), 1.52-1.26 (m, 13H), 1.22 (s, 3H), 1.06 (dd, J=2.1, 12.6 Hz, 2H), 1.02-1.00 (m, 3H), 0.99 (s, 3H), 0.93-0.90 (m, 3H), 0.89 (s, 3H), 0.82 (s, 3H), 0.70 (s, 3H);

Mass spectrum: (ESI, positive) m/z 445.2 [M+H]⁺.

### Step 3: Synthesis of Compound 1

DMAP (5.49 g, 44.9 mmol) and compound e (8.65 g, 44.9 mmol) were added to a solution of compound c (5.00 g, 11.2 mmol) in DCM (50.0 mL), and the reaction mixture was stirred at 25° C. for 24 hours. H₂O (50.0 mL) was added to the reaction mixture and the reaction mixture was extracted with DCM (50.0 mL*3). The organic phase was washed with brine (100 mL), dried over anhydrous sodium sulfate, and then concentrated under reduced pressure to obtain a residue. The residue was purified by column chromatography (petroleum ether/ethyl acetate=100/1 to 1/1) to obtain compound 1 (1.50 g, 2.35 mmol, 21.1% yield).

¹H NMR (400 MHZ, CDCl₃) δ=4.55 (dd, J=4.6, 11.4 Hz, 1H), 2.95 (q, J=6.7 Hz, 4H), 2.83-2.73 (m, 4H), 2.09-1.98 (m, 4H), 1.96-1.87 (m, 1H), 1.80-1.64 (m, 5H), 1.63-1.50 (m, 3H), 1.49-1.15 (m, 18H), 1.01 (s, 3H), 0.93-0.87 (m, 12H), 0.72-0.68 (m, 3H).

Mass spectrum: (ESI, positive) m/z 636.9 [M+H]⁺.

### Example 2

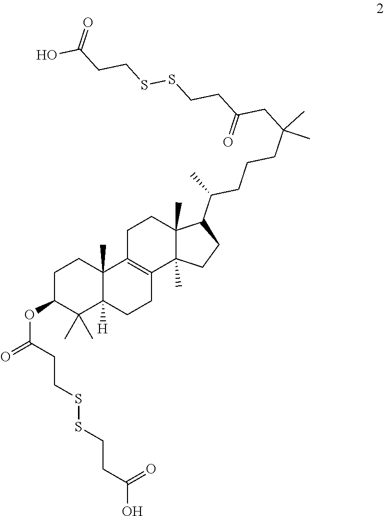

### Synthetic Route

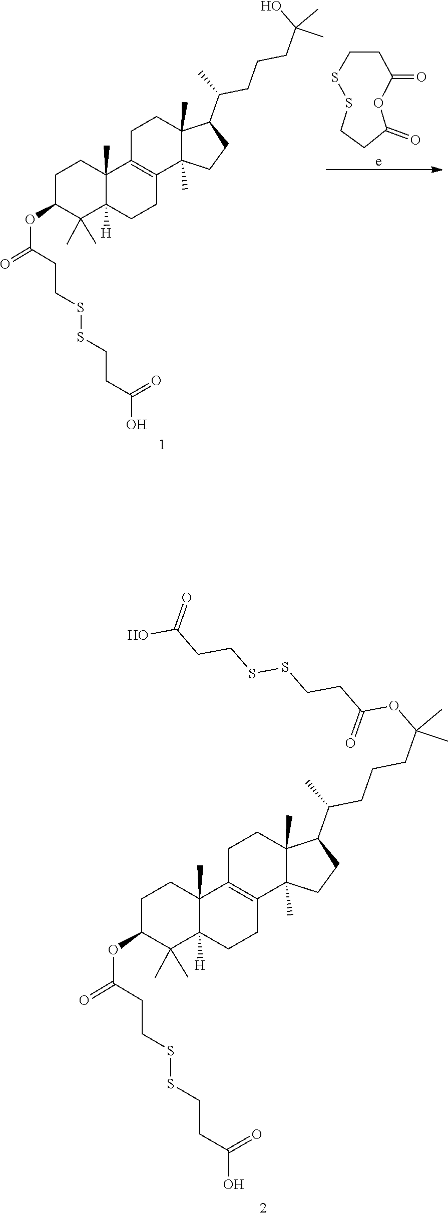

### Synthesis of Compound 2:

Compound e (0.865 g, 4.49 mmol) was added to a solution of compound 1 (0.75 g, 1.178 mmol) in pyridine (50.0 mL). The reaction mixture was stirred at 90° C. for 14 hours. H₂O (50.0 mL) was added to the reaction mixture and extracted with DCM (50.0 mL*3). The organic phase was washed with brine (100 mL), dried over anhydrous sodium sulfate, and then concentrated under reduced pressure to obtain a residue. The residue was purified by column chromatography (petroleum ether/ethyl acetate=100/1 to 1/1) to obtain compound 2 (0.36 g, 0.435 mmol, 36.9% yield).

¹H NMR (400 MHZ,) δ=4.56-4.52 (m, 1H), 2.91-2.86 (m, 8H), 2.75-2.70 (m, 6H), 2.66-2.63 (m, 2H), 2.16-2.00 (m, 2H), 1.99-1.78 (m, 6H), 1.76-1.48 (m, 8H), 1.46 (s, 6H), 1.44-1.24 (m, 4H), 1.22-1.10 (m, 4H), 0.98-0.94 (m, 9H), 0.93-0.88 (m, 4H), 0.87 (s, 3H), 0.68 (s, 3H).

Mass spectrum: (ESI, positive) m/z 829.1 [M+H]⁺.

### Example 3-44

The following compounds were synthesized referring to the above-mentioned synthesis method:

| Example | Example 3 |
|---|---|
| Compound | 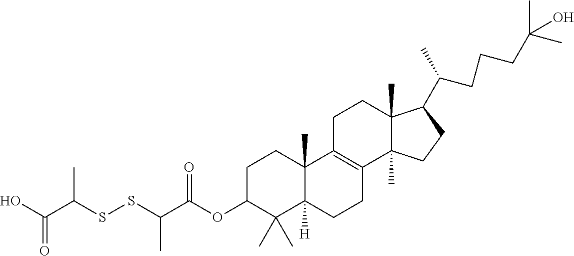 [SMILES: CC(SSC(C)C(=O)OC1CC[C@]2(C)C3=C(CC[C@H]2C1(C)C)[C@]1(C)CC[C@H]([C@H](C)CCCC(C)(C)O)[C@@]1(C)CC3)C(=O)O] |
| [M + H]⁺ | 636.8 |
| Example | Example 4 |
| Compound | 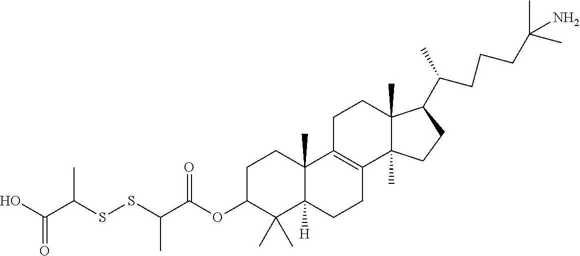 [SMILES: CC(SSC(C)C(=O)OC1CC[C@]2(C)C3=C(CC[C@H]2C1(C)C)[C@]1(C)CC[C@H]([C@H](C)CCCC(C)(C)N)[C@@]1(C)CC3)C(=O)O] |
| [M + H]⁺ | 635.9 |

| Example | Example 5 |
|---|---|
| Compound | 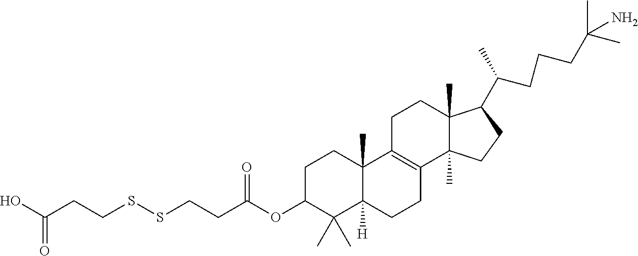 [SMILES: C[C@H](CCCC(C)(C)N)[C@H]1CC[C@@]2(C)C3=C(CC[C@]12C)[C@@]1(C)CCC(OC(=O)CCSSCCC(=O)O)C(C)(C)[C@@H]1CC3] |
| [M + H]⁺ | 636.1 |
| Example | Example 6 |
| Compound | 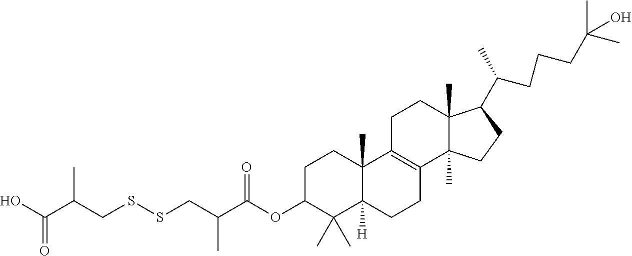 [SMILES: CC(CSSCC(C)C(=O)OC1CC[C@]2(C)C3=C(CC[C@H]2C1(C)C)[C@]1(C)CC[C@H]([C@H](C)CCCC(C)(C)O)[C@@]1(C)CC3)C(=O)O] |
| [M + H]⁺ | 665.3 |

| Example | Example 7 |
|---|---|
| Compound | 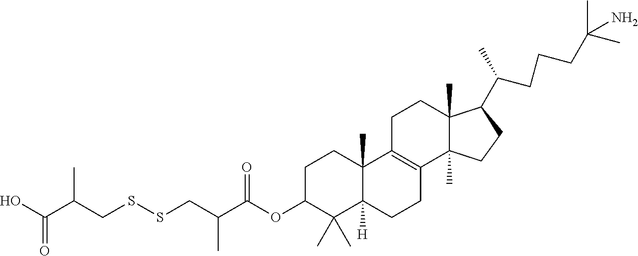 [SMILES: CC(CSSCC(C)C(=O)OC1CC[C@]2(C)C3=C(CC[C@H]2C1(C)C)[C@]1(C)CC[C@H]([C@H](C)CCCC(C)(C)N)[C@@]1(C)CC3)C(=O)O] |
| [M + H]⁺ | 664.2 |
| Example | Example 8 |
| Compound | 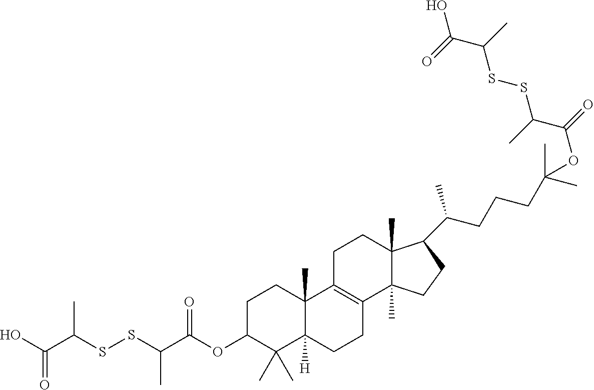 [SMILES: CC(SSC(C)C(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3)C(=O)O] |
| [M + H]⁺ | 828.9 + H |

| Example | Example 9 |
|---|---|
| Compound | 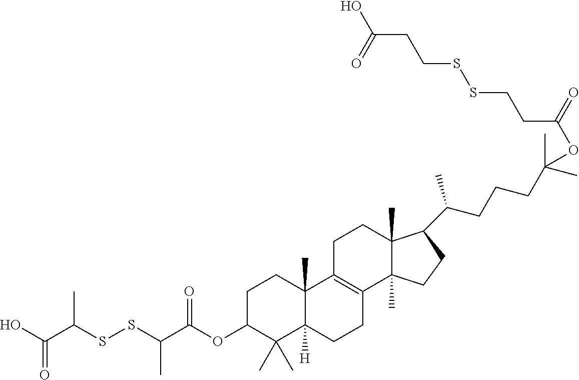 [SMILES: CC(SSC(C)C(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3)C(=O)O] |
| [M + H]⁺ | 828.8 |
| Example | Example 10 |
| Compound | 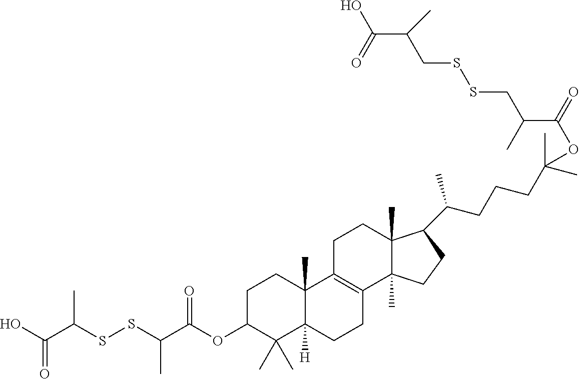 [SMILES: CC(CSSCC(C)C(=O)OC(C)(C)CCC[C@@H](C)[C@H]1CC[C@@]2(C)C3=C(CC[C@]12C)[C@@]1(C)CCC(OC(=O)C(C)SSC(C)C(=O)O)C(C)(C)[C@@H]1CC3)C(=O)O] |
| [M + H]⁺ | 857.2 |

| Example | Example 11 | Example 12 |
|---|---|---|
| Com- pound | 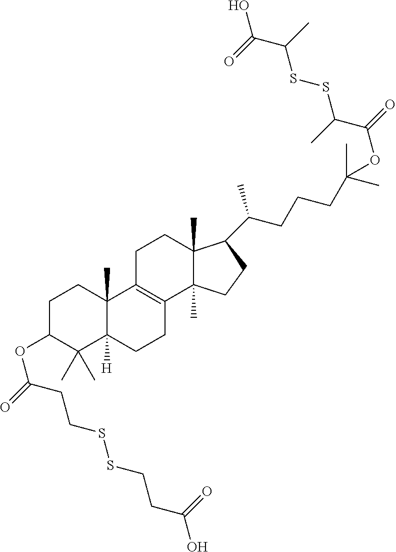 [SMILES: CC(SSC(C)C(=O)OC(C)(C)CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3)C(=O)O] | 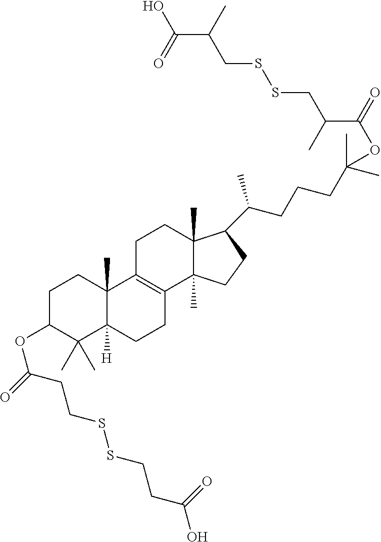 [SMILES: CC(CSSCC(C)C(=O)OC(C)(C)CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3)C(=O)O] |
| [M + H]⁺ | 829.3 | 856.9 |

| Example | Example 13 |
|---|---|
| Compound | 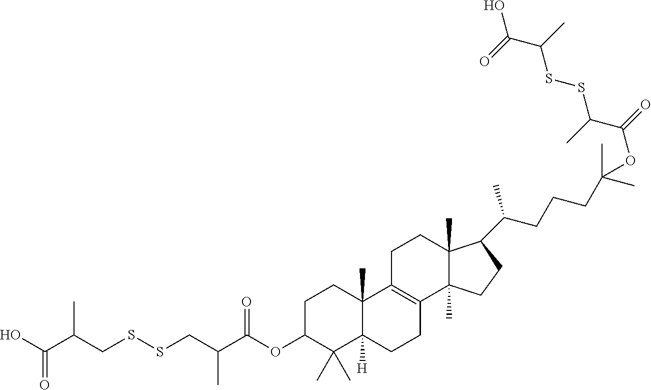 [SMILES: CC(CSSCC(C)C(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3)C(=O)O] |
| [M + H]⁺ | 857.1 |
| Example | Example 14 |
| Compound | 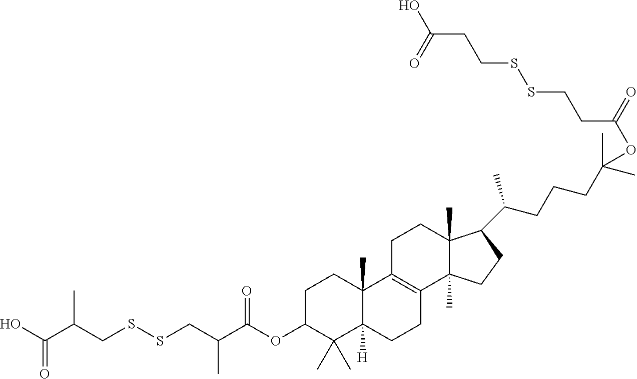 [SMILES: CC(CSSCC(C)C(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3)C(=O)O] |
| [M + H]⁺ | 856.9 |

| Example | Example 15 |
|---|---|
| Compound | 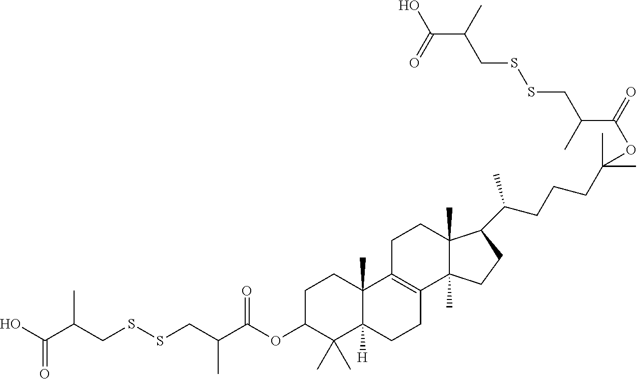 [SMILES: CC(CSSCC(C)C(=O)OC1CC[C@]2(C)C3=C(CC[C@H]2C1(C)C)[C@]1(C)CC[C@H]([C@H](C)CCCC(C)(C)OC(=O)C(C)CSSCC(C)C(=O)O)[C@@]1(C)CC3)C(=O)O] |
| [M + H]⁺ | 885.2 |
| Example | Example 16 |
| Compound | 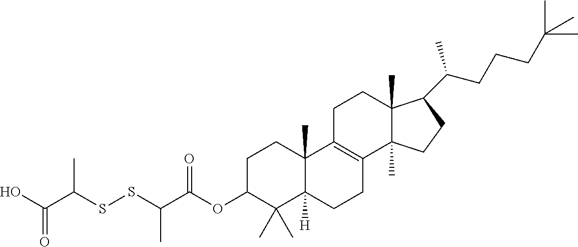 [SMILES: CC(SSC(C)C(=O)OC1CC[C@]2(C)C3=C(CC[C@H]2C1(C)C)[C@]1(C)CC[C@H]([C@H](C)CCCC(C)(C)C)[C@@]1(C)CC3)C(=O)O] |
| [M + H]⁺ | 635.3 |

| Example | Example 17 |
|---|---|
| Compound | 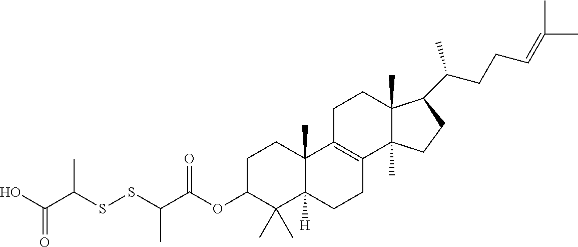 [SMILES: CC(C)=CCC[C@@H](C)[C@H]1CC[C@@]2(C)C3=C(CC[C@]12C)[C@@]1(C)CCC(OC(=O)C(C)SSC(C)C(=O)O)C(C)(C)[C@@H]1CC3] |
| [M + H]⁺ | 618.9 |
| Example | Example 18 |
| Compound | 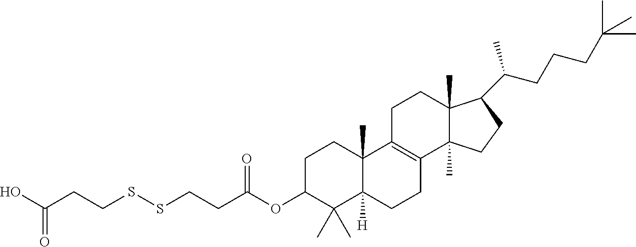 [SMILES: C[C@H](CCCC(C)(C)C)[C@H]1CC[C@@]2(C)C3=C(CC[C@]12C)[C@@]1(C)CCC(OC(=O)CCSSCCC(=O)O)C(C)(C)[C@@H]1CC3] |
| [M + H]⁺ | 635.1 |

| Example | Example 19 |
|---|---|
| Compound | 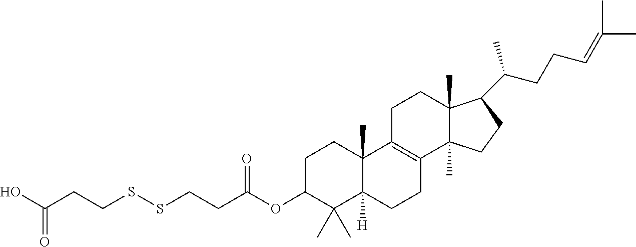 [SMILES: CC(C)=CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3] |
| [M + H]⁺ | 618.9 |
| Example | Example 20 |
| Compound | 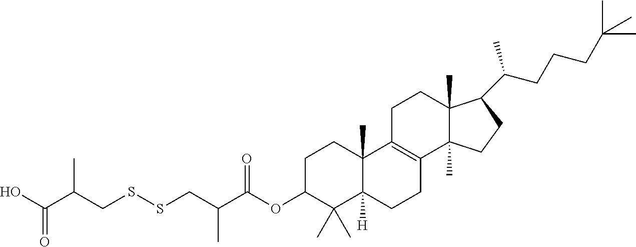 [SMILES: CC(CSSCC(C)C(=O)OC1CC[C@]2(C)C3=C(CC[C@H]2C1(C)C)[C@]1(C)CC[C@H]([C@H](C)CCCC(C)(C)C)[C@@]1(C)CC3)C(=O)O] |
| [M + H]⁺ | 663.1 |

| Example | Example 21 |
|---|---|
| Compound | 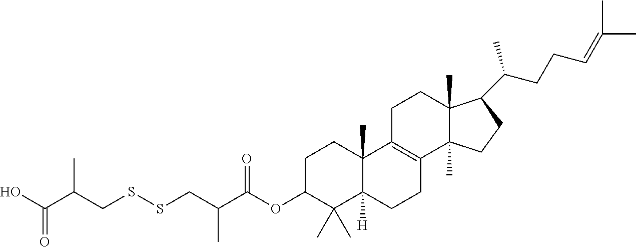 [SMILES: CC(C)=CCC[C@@H](C)[C@H]1CC[C@@]2(C)C3=C(CC[C@]12C)[C@@]1(C)CCC(OC(=O)C(C)CSSCC(C)C(=O)O)C(C)(C)[C@@H]1CC3] |
| [M + H]⁺ | 647.3 |
| Example | Example 22 |
| Compound | 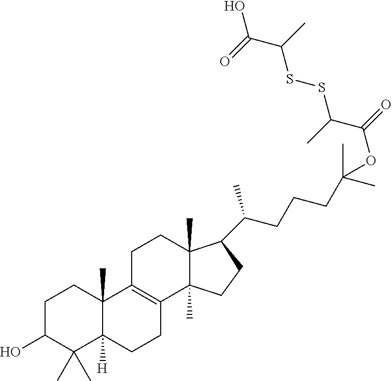 [SMILES: CC(SSC(C)C(=O)OC(C)(C)CCC[C@@H](C)[C@H]1CC[C@@]2(C)C3=C(CC[C@]12C)[C@@]1(C)CCC(O)C(C)(C)[C@@H]1CC3)C(=O)O] |
| [M + H]⁺ | 637.2 |

| Example | Example 23 |
|---|---|
| Compound | 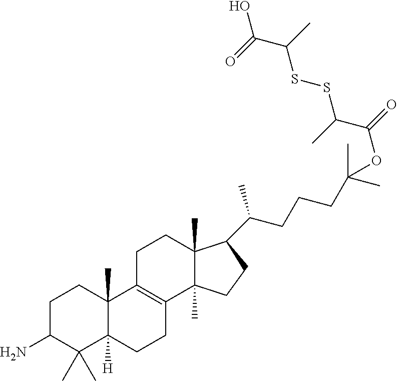 [SMILES: CC(SSC(C)C(=O)OC(C)(C)CCC[C@@H](C)[C@H]1CC[C@@]2(C)C3=C(CC[C@]12C)[C@@]1(C)CCC(N)C(C)(C)[C@@H]1CC3)C(=O)O] |
| [M + H]⁺ | 636.2 |
| Example | Example 24 |
| Compound | 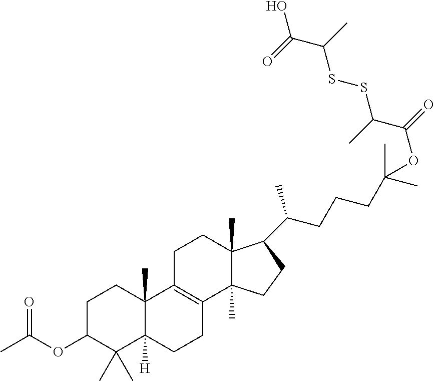 [SMILES: CC(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3] |
| [M + H]⁺ | 679.1 |

| Example | Example 25 | Example 26 |
|---|---|---|
| Compound | 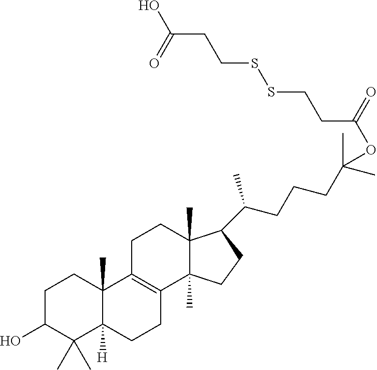 [SMILES: C[C@H](CCCC(C)(C)OC(=O)CCSSCCC(=O)O)[C@H]1CC[C@@]2(C)C3=C(CC[C@]12C)[C@@]1(C)CCC(O)C(C)(C)[C@@H]1CC3] | 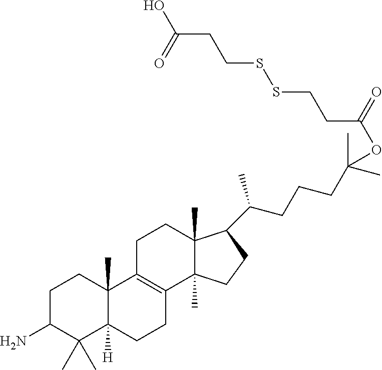 [SMILES: C[C@H](CCCC(C)(C)OC(=O)CCSSCCC(=O)O)[C@H]1CC[C@@]2(C)C3=C(CC[C@]12C)[C@@]1(C)CCC(N)C(C)(C)[C@@H]1CC3] |
| [M + H]⁺ | 636.9 | 636.3 |

| Example | Example 27 | Example 28 |
|---|---|---|
| Com- pound | 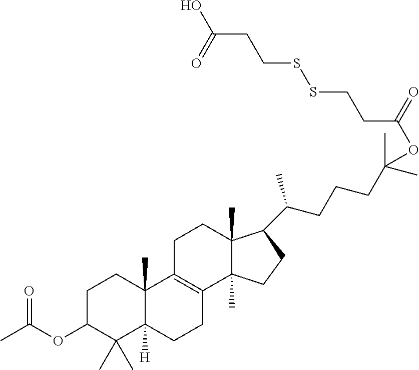 [SMILES: CC(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3] | 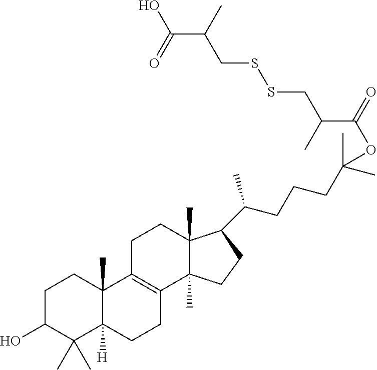 [SMILES: CC(CSSCC(C)C(=O)OC(C)(C)CCC[C@@H](C)[C@H]1CC[C@@]2(C)C3=C(CC[C@]12C)[C@@]1(C)CCC(O)C(C)(C)[C@@H]1CC3)C(=O)O] |
| [M + H]⁺ | 679.1 | 664.9 |

| Example | Example 29 | Example 30 |
|---|---|---|
| Com- pound | 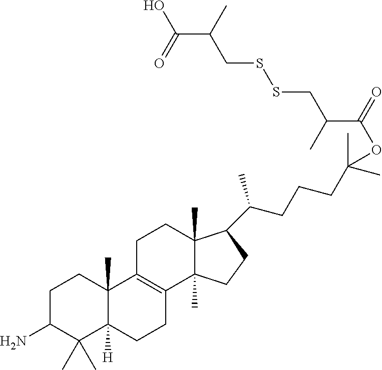 [SMILES: CC(CSSCC(C)C(=O)OC(C)(C)CCC[C@@H](C)[C@H]1CC[C@@]2(C)C3=C(CC[C@]12C)[C@@]1(C)CCC(N)C(C)(C)[C@@H]1CC3)C(=O)O] | 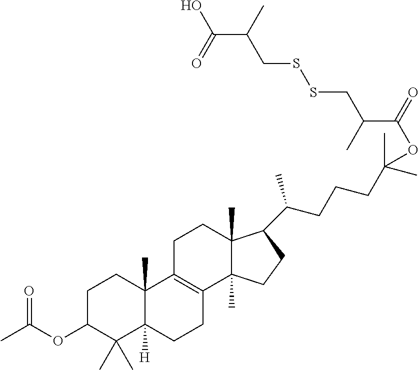 [SMILES: CC(=O)OC1CC[C@]2(C)C3=C(CC[C@H]2C1(C)C)[C@]1(C)CC[C@H]([C@H](C)CCCC(C)(C)OC(=O)C(C)CSSCC(C)C(=O)O)[C@@]1(C)CC3] |
| [M + H]⁺ | 664.3 | 707.1 |

| Example | Example 31 |
|---|---|
| Compound | 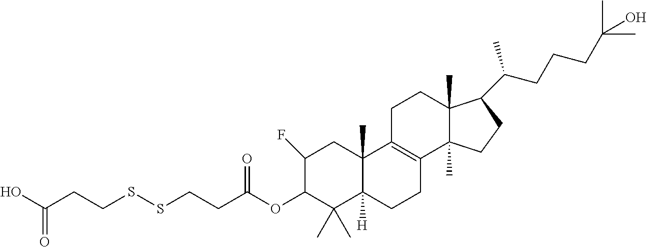 [SMILES: C[C@H](CCCC(C)(C)O)[C@H]1CC[C@@]2(C)C3=C(CC[C@]12C)[C@@]1(C)CC(F)C(OC(=O)CCSSCCC(=O)O)C(C)(C)[C@@H]1CC3] |
| [M + H]⁺ | 654.9 |
| Example | Example 32 |
| Compound | 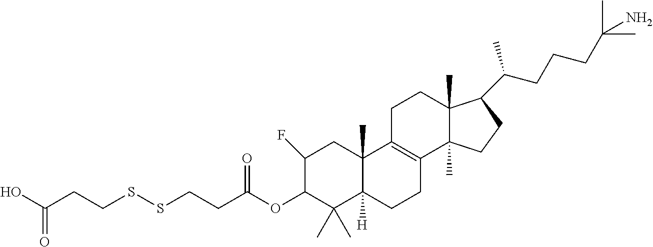 [SMILES: C[C@H](CCCC(C)(C)N)[C@H]1CC[C@@]2(C)C3=C(CC[C@]12C)[C@@]1(C)CC(F)C(OC(=O)CCSSCCC(=O)O)C(C)(C)[C@@H]1CC3] |
| [M + H]⁺ | 653.9 |

| Example | Example 33 |
|---|---|
| Com- pound | 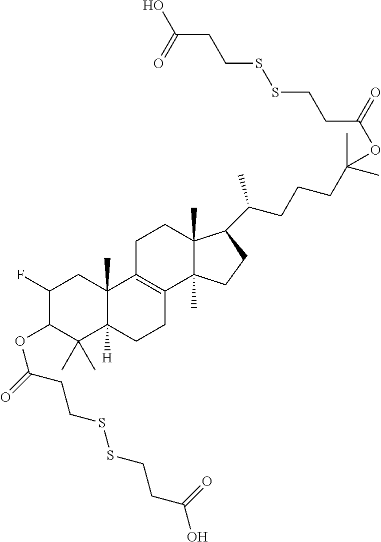 [SMILES: C[C@H](CCCC(C)(C)OC(=O)CCSSCCC(=O)O)[C@H]1CC[C@@]2(C)C3=C(CC[C@]12C)[C@@]1(C)CC(F)C(OC(=O)CCSSCCC(=O)O)C(C)(C)[C@@H]1CC3] |
| [M + H]⁺ | 847.2 |
| Example | Example 34 |
| Com- pound | 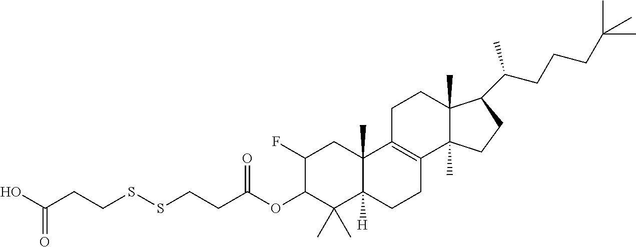 [SMILES: C[C@H](CCCC(C)(C)C)[C@H]1CC[C@@]2(C)C3=C(CC[C@]12C)[C@@]1(C)CC(F)C(OC(=O)CCSSCCC(=O)O)C(C)(C)[C@@H]1CC3] |
| [M + H]⁺ | 652.9 |

| Example | Example 35 |
|---|---|
| Com- pound | 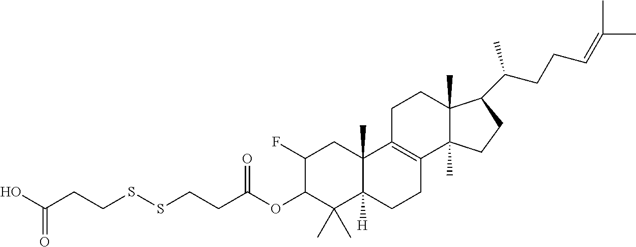 [SMILES: CC(C)=CCC[C@@H](C)[C@H]1CC[C@@]2(C)C3=C(CC[C@]12C)[C@@]1(C)CC(F)C(OC(=O)CCSSCCC(=O)O)C(C)(C)[C@@H]1CC3] |
| [M + H]⁺ | 637.1 |
| Example | Example 36 |
| Com- pound | 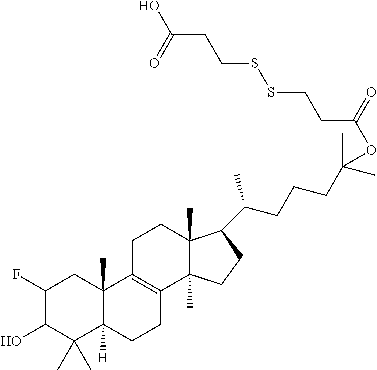 [SMILES: C[C@H](CCCC(C)(C)OC(=O)CCSSCCC(=O)O)[C@H]1CC[C@@]2(C)C3=C(CC[C@]12C)[C@@]1(C)CC(F)C(O)C(C)(C)[C@@H]1CC3] |
| [M + H]⁺ | 654.9 |

| Example | Example 37 | Example 38 |
|---|---|---|
| Com- pound | 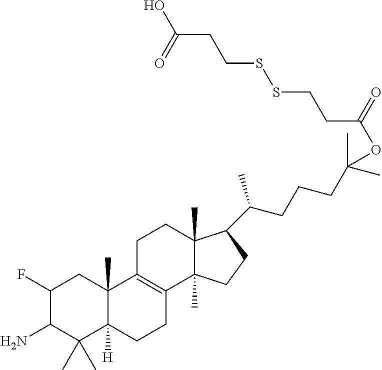 [SMILES: C[C@H](CCCC(C)(C)OC(=O)CCSSCCC(=O)O)[C@H]1CC[C@@]2(C)C3=C(CC[C@]12C)[C@@]1(C)CC(F)C(N)C(C)(C)[C@@H]1CC3] | 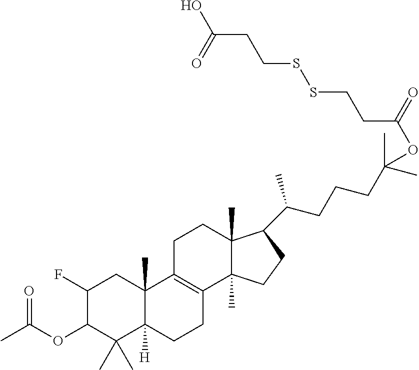 [SMILES: CC(=O)OC1C(F)C[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3] |
| [M + H]⁺ | 654.1 | 696.9 |

| Example | Example 39 |
|---|---|
| Com- pound | 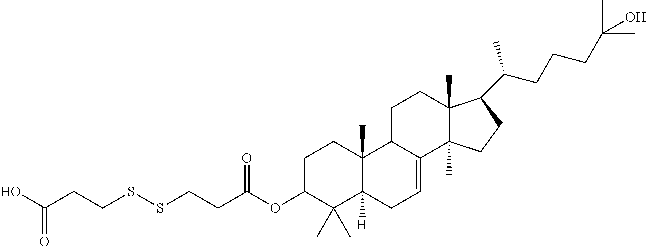 [SMILES: C[C@H](CCCC(C)(C)O)[C@H]1CC[C@@]2(C)C3=CC[C@H]4C(C)(C)C(OC(=O)CCSSCCC(=O)O)CC[C@]4(C)C3CC[C@]12C] |
| [M + H]⁺ | 637.1 |
| Example | Example 40 |
| Com- pound | 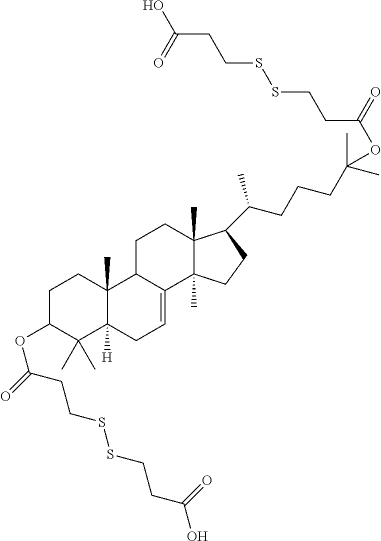 [SMILES: C[C@H](CCCC(C)(C)OC(=O)CCSSCCC(=O)O)[C@H]1CC[C@@]2(C)C3=CC[C@H]4C(C)(C)C(OC(=O)CCSSCCC(=O)O)CC[C@]4(C)C3CC[C@]12C] |
| [M + H]⁺ | 828.9 |

| Example | Example 41 |
|---|---|
| Com- pound | 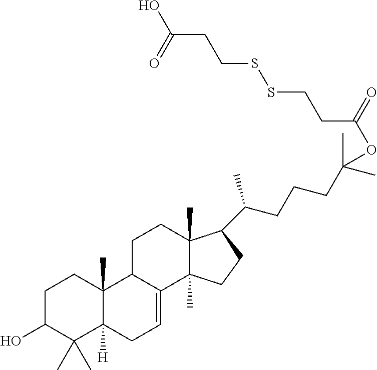 [SMILES: C[C@H](CCCC(C)(C)OC(=O)CCSSCCC(=O)O)[C@H]1CC[C@@]2(C)C3=CC[C@H]4C(C)(C)C(O)CC[C@]4(C)C3CC[C@]12C] |
| [M + H]⁺ | 636.9 |
| Example | Example 42 |
| Com- pound | 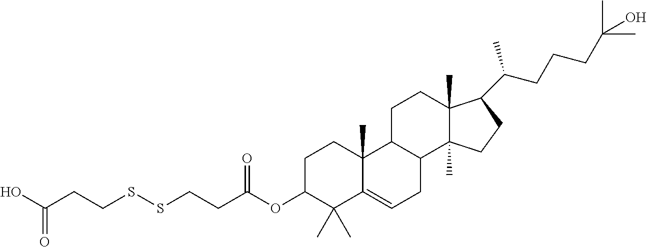 [SMILES: C[C@H](CCCC(C)(C)O)[C@H]1CC[C@@]2(C)C3CC=C4C(C)(C)C(OC(=O)CCSSCCC(=O)O)CC[C@]4(C)C3CC[C@]12C] |
| [M + H]⁺ | 637.3 |

| Example | Example 43 | Example 44 |
|---|---|---|
| Compound | 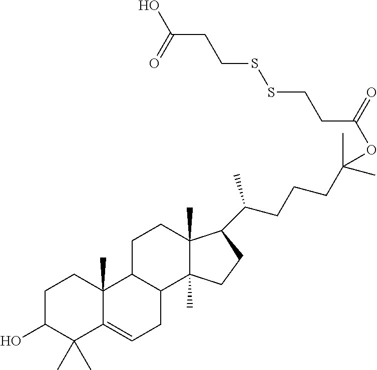 [SMILES: C[C@H](CCCC(C)(C)OC(=O)CCSSCCC(=O)O)[C@H]1CC[C@@]2(C)C3CC=C4C(C)(C)C(O)CC[C@]4(C)C3CC[C@]12C] | 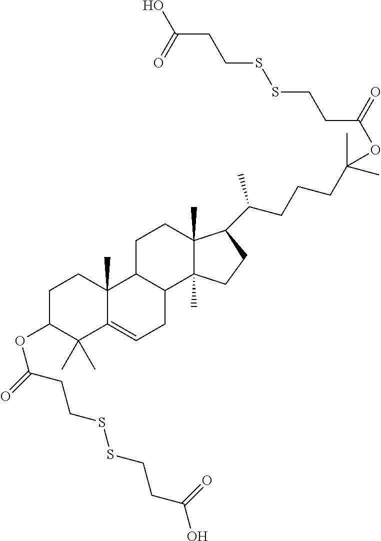 [SMILES: C[C@H](CCCC(C)(C)OC(=O)CCSSCCC(=O)O)[C@H]1CC[C@@]2(C)C3CC=C4C(C)(C)C(OC(=O)CCSSCCC(=O)O)CC[C@]4(C)C3CC[C@]12C] |
| [M + H]⁺ | 636.9 | 829.1 |

### Biological Activity Assay

### Experiment I: Pharmacodynamic Study on Sodium Selenite-Induced New Zealand Rabbit Cataract Model

### 1. EXPERIMENTAL ANIMALS

Neonatal New Zealand rabbits were P7 days old in ordinary grade, and 5 baby rabbits per litter were breastfed with one mother rabbit.

### 2. GROUPING AND PROCESSING

The young rabbits were randomly divided into 6 groups with 5 rabbits in each group.
1) Normal control group (CG): Normal feeding.
2) Disease model group (MG): At day P10, young rabbits were injected subcutaneously with sodium selenite solution (in normal saline) at 20 μmol/kg body weight. After day P15, normal saline eye drops were dripped into the left eye 3 times a day for 30 consecutive days.
3) Compound 1 group (group 1, G1): At day P10, young rabbits were injected subcutaneously with sodium selenite solution (in normal saline) at 20 μmol/kg body weight. After day P15, 1.5% (w/w) compound 1 eye drops were dripped into the left eye 3 times a day for 30 consecutive days.
4) Compound 2 group (group 2, G2): At day P10, young rabbits were injected subcutaneously with sodium selenite solution (in normal saline) at 20 μmol/kg body weight of. After day P15, 1.5% (w/w) compound 2 eye drops were dripped into the left eye 3 times a day for 30 consecutive days.
5) Compound c group (group c, Gc): At day P10, young rabbits were injected subcutaneously with sodium selenite solution (in normal saline) at 20 μmol/kg body weight. After day P15, 1.5% (w/w) compound c eye drops were dripped into the left eye 3 times a day for 30 consecutive days.
6) 3,3′-dithiodipropionic acid group (group d, Gd): At day P10, young rabbits were injected subcutaneously with sodium selenite solution (in normal saline) at 20 μmol/kg body weight. After day P15, 1.5% (w/w) 3,3′-dithiodipropionic acid eye drops were dripped into the left eye 3 times a day for 30 consecutive days.

### 3. EXPERIMENTAL TESTING

Slit lamp observation: Slit lamp observation was performed on each group of sodium selenite-induced neonatal New Zealand rabbits before the administration, and 7 days, 14 days, and 30 days after the administration respectively, and the degree of lens opacity was graded to determine the therapeutic effect. Lens opacity grading standard [Hiraoka T, Clark J I. Inhibition of lens opacification during the early stages of cataract formation[J]. Invest Ophthalmol Vis Sci, 1995, 36(12):2550-5.]: Grade 0: transparent lens: Grade I: vacuoles can be seen in the periphery or anterior cortex of the lens: Grade II: slightly increased density of the lens nucleus: Grade III: translucent lens nucleus: Grade IV: opacity of the lens nucleus but not involving the cortex: Grade V: opacity of the lens nucleus and perinuclear cortex: Grade VI: complete opacity of the lens.

### 4. EXPERIMENTAL RESULTS

| | | | | | | | | |
|---|---|---|---|---|---|---|---|---|
| Groups | Lens number | I | II | III | IV | V | VI | VII |

| | | | | | | | |
|---|---|---|---|---|---|---|---|
| CG | 5 | | | | | | |
| MG | 5 | | | | | 1 | 4 |
| G1 | 5 | 1 | 2 | 1 | 1 | | |
| G2 | 5 | 3 | 1 | 1 | | | |
| Gc | 5 | | 2 | 3 | | | |
| Gd | 5 | | | 1 | 2 | 1 | 1 |

The experimental results are shown in the table, and a chi-square test was performed on the significance between the results of various groups. The effect of group G1 was significantly superior to that of the model group (MG) (p<0.05); the effect of group G1 was significantly superior to that of the core fragment compound c (Gc) group (p<0.05); the effect of group G1 was significantly superior to that of the core fragment compound d (Gd) group (p<0.05). The effect of group G2 was significantly superior to that of the model group (MG) (p<0.05); the effect of group G2 was significantly superior to that of the core fragment compound c (Gc) group (p<0.05): the effect of group G2 was significantly superior to that of the core fragment compound d (Gd) group (p<0.05).

### 5. CONCLUSION

The above results suggest that compounds 1 and 2 have significant therapeutic effects on the sodium selenite-induced cataract model, and compound 2 has the best therapeutic effect.

### Experiment II: Pharmacodynamic Study on Mice Presbyopia Model

### 1. EXPERIMENTAL ANIMALS

Mice (8 months old, C57BL/6J), normal grade.

### 2. GROUPING AND PROCESSING

The mice were randomly divided into 4 groups with 5 mice in each group.
1) Model group (MG): normal saline eye drops were dripped into the left eye for 45 consecutive days, 3 times a day.
2) Compound 1 Group (group 1, G1): 1.5% (w/w) compound 1 eye drops were dripped into the left eye for 45 consecutive days, 3 times a day.
3) Compound 2 Group (group 2, G2): 1.5% (w/w) compound 2 eye drops were dripped into the left eye for 45 consecutive days, 3 times a day.
4) 3,3′-dithiodipropionic acid group (group d, Gd): 1.5% (w/w) 3,3′-dithiodipropionic acid eye drops were dripped into the left eye for 45 consecutive days, 3 times a day.

### 3. EXPERIMENTAL TESTING

Measurement of lens thickness deformation: After 45 consecutive days of eye dripping, mice were euthanized with carbon dioxide and flushed with saline. Lens thickness was measured using a micrometer screw after applying a fixed pressure.

### 4. EXPERIMENTAL RESULTS

| | | Lens thickness |
|---|---|---|
| Groups | Lens number | (deformation) |
| MG | 5 | 1.65 ± 0.02 |
| G1 | 5 | 1.42 ± 0.03 |
| G2 | 5 | 1.51 ± 0.02 |
| Gd | 5 | 1.55 ± 0.01 |

### 5. CONCLUSION

The experimental results are shown in the table. Through one-way four-level t-test, the differences between each group and the MG group were statistically significant (p<0.05).

The above results suggest that compounds 1 and 2 of the present invention have significant therapeutic effects on elastic recovery of aged mice, and group G1 has the most significant effect on elastic recovery.

Although the present application has been disclosed as above with preferred examples, any person skilled in the art may make several possible changes and modifications without departing from the concept of the present application. Therefore, the protective scope of the present application shall be based on the scope defined by the claims of the present application.

1. A compound represented by formula (I) or (II), or a pharmaceutically acceptable salt or isomer thereof: 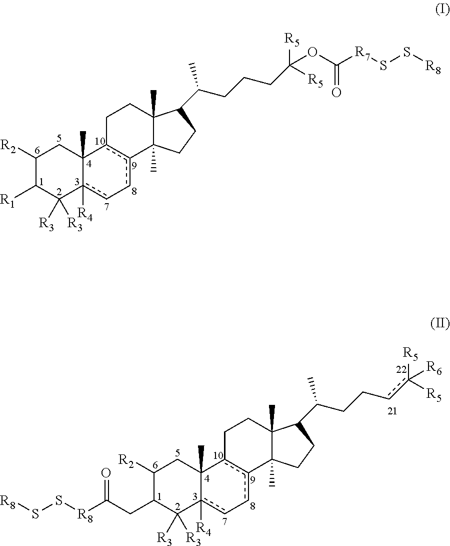
wherein,
R₁ is selected from the group consisting of OH, NH₂, and 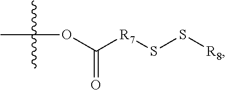
or R₁ is selected from the group consisting of C₁₋₆ alkyl, —OC₁₋₆ alkyl, —OC(═O)C₁₋₆ alkyl, C₁₋₆ heteroalkyl, —OC₁₋₆ heteroalkyl, —OC(═O)C₁₋₆ heteroalkyl, C₃₋₈ cycloalkyl, —OC₃₋₈ cycloalkyl, —OC(═O)C₃₋₈ cycloalkyl, 5-10 membered heterocycloalkyl, C₆₋₁₀ aryl, and 5-10 membered heteroaryl, which are unsubstituted or substituted by 1, 2 or 3 Rs;
R₂ is selected from the group consisting of H, F, Cl, Br, I, NH₂, OH, COOH, and CONH₂;
R₃ is selected from the group consisting of H, F, Cl, Br, I, NH₂, and OH, or R₃ is selected from the group consisting of C₁₋₆ alkyl unsubstituted or substituted by 1, 2 or 3 Rs;
the “ [symbol] ” between carbon atoms No. 3 and 7 is a single or double bond, and when it is a single bond, R₄ is H; and when it is a double bond, R₄ is absent;
the “ [symbol] ” between adjacent carbon atoms No. 8, 9 and 10 is a single or double bond, and the two bonds cannot be double bonds simultaneously;
R₅ is independently selected from the group consisting of H, and C₁₋₆ alkyl unsubstituted or substituted by 1, 2 or 3 Rs;
the “ [symbol] ” between carbon atoms No. 21 and 22 is a single or double bond, and when it is a single bond, R₆ is selected from the group consisting of OH, NH₂, and 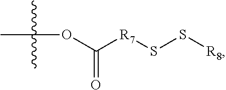
or R₆ is selected from the group consisting of C₁₋₆ alkyl, —OC₁₋₆ alkyl, —OC(═O)C₁₋₆ alkyl, C₁₋₆ heteroalkyl, —OC₁₋₆ heteroalkyl, —OC(═O)C₁₋₆ heteroalkyl, C₃₋₈ cycloalkyl, —OC₃₋₈ cycloalkyl, —OC(═O)C₃₋₈ cycloalkyl, 5-10 membered heterocycloalkyl, C₆₋₁₀ aryl, and 5-10 membered heteroaryl, which are unsubstituted or substituted by 1, 2 or 3 Rs; and when it is a double bond, R₆ is absent;
R₇ is selected from the group consisting of C₁₋₁₂ alkyl and C₁₋₁₂ heteroalkyl, which are unsubstituted or substituted by 1, 2, or 3 Rs;
R₈ is selected from the group consisting of C₁₋₁₂ alkyl and C₁₋₁₂ heteroalkyl, which are unsubstituted or substituted by 1, 2, 3, or 4 Rs;
Rs are independently selected from the group consisting of F, Cl, Br, I, NH₂, OH, COOH, CONH₂, C16 alkyl, C₂₋₆ alkenyl, and C₂₋₆ alkynyl;
the “hetero” in the heteroalkyl, heterocycloalkyl, and heteroaryl is independently selected from the following heteroatoms or heteroradicals: —NH—, N, —O—, and —S—; and the number of the heteroatom or heteroradical is independently selected from 1, 2, and 3.

2. The compound according to claim 1, or a pharmaceutically acceptable salt or isomer thereof, wherein Rs are each independently selected from the group consisting of F, Cl, Br, I, NH₂, OH, COOH, CONH₂, CH₃, —CH₂CH₃, —(CH₂)₂CH₃, —(CH₂)₃CH₃, —CH═CH₂, and —CH═CH—CH₂.

3. The compound according to claim 1, or a pharmaceutically acceptable salt or isomer thereof, wherein R₁ is selected from the group consisting of OH, NH₂, and 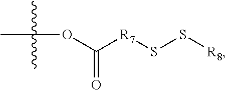
or R₁ is selected from the group consisting of 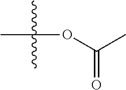 [SMILES: *OC(C)=O]
unsubstituted or substituted by 1, 2, or 3 Rs,
wherein, R₇ is selected from the group consisting of C₁₋₆ alkyl and —NH—C₁₋₆ alkyl-, which are unsubstituted or substituted by 1, 2, or 3 Rs;
R₈ is selected from the group consisting of C₁₋₆ alkyl and —C₁₋₆ alkyl-NH₂, which are unsubstituted or substituted by 1, 2, 3, or 4 Rs.

4. The compound according to claim 1, or a pharmaceutically acceptable salt or isomer thereof, wherein R₁ is selected from the group consisting of OH, NH₂, and 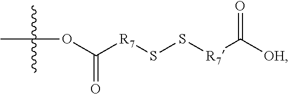
wherein, R₇ and R₇′ are each independently selected from the group consisting of C₁₋₆ alkyl unsubstituted or substituted by 1, 2, or 3 Rs, preferably methyl, ethyl and propyl, which are unsubstituted or substituted by 1, 2, or 3 Rs.

5. The compound according to claim 1, or a pharmaceutically acceptable salt or isomer thereof, wherein R₁ is selected from the group consisting of:

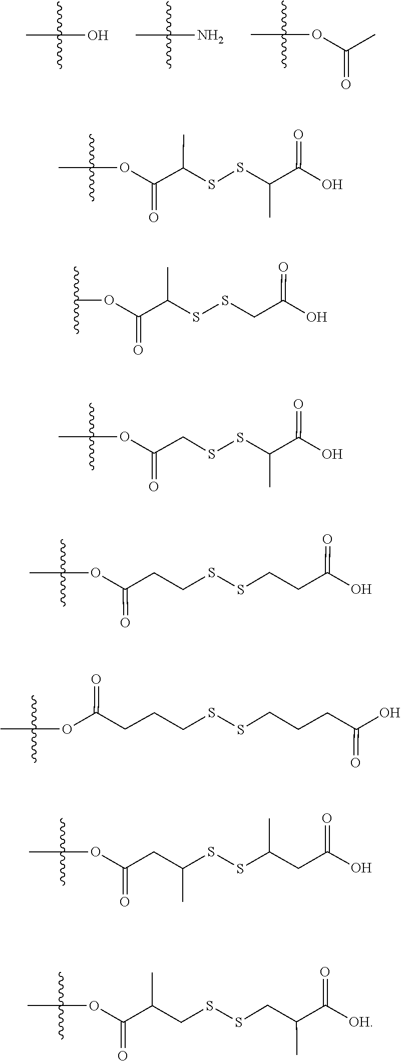

6. The compound according to claim 1, or a pharmaceutically acceptable salt or isomer thereof, wherein R₂ is selected from the group consisting of H, F, Cl, NH₂, OH, COOH, and CONH₂.

7. The compound according to claim 1, or a pharmaceutically acceptable salt or isomer thereof, wherein R₃ is selected from the group consisting of H, F, Cl, Br, I, NH₂, OH, CH₃, —CH₂OH, —CH₂CH₃, —(CH₂)₂OH, and —(CH₂)₃CH₃.

8. The compound according to claim 1, or a pharmaceutically acceptable salt or isomer thereof, wherein Rs is selected from the group consisting of H, CH₃, —CH₂OH, —CH₂CH₃, and —(CH₂)₂OH.

9. The compound according to claim 1, or a pharmaceutically acceptable salt or isomer thereof, wherein R₆ is selected from the group consisting of OH, NH₂, and 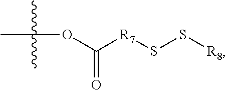
or R₆ is selected from the group consisting of 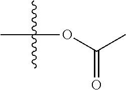 [SMILES: *OC(C)=O]
unsubstituted or substituted by 1, 2, or 3 Rs,
wherein, R₇ is selected from the group consisting of C₁₋₆ alkyl and —NH—C₁₋₆ alkyl-, which are unsubstituted or substituted by 1, 2, or 3 Rs;
R₈ is selected from C₁₋₆ alkyl and —C₁₋₆ alkyl-NH₂, which are unsubstituted or substituted by 1, 2, 3, or 4 Rs.

10. The compound according to claim 1, or a pharmaceutically acceptable salt or isomer thereof, wherein R₆ is selected from the group consisting of OH, NH₂, and 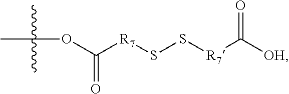
wherein, R₇ and R₇′ are independently selected from the group consisting of C₁₋₆ alkyl unsubstituted or substituted by 1, 2, or 3 Rs.

11. The compound according to claim 1, or a pharmaceutically acceptable salt or isomer thereof, wherein R₆ is selected from the group consisting of:

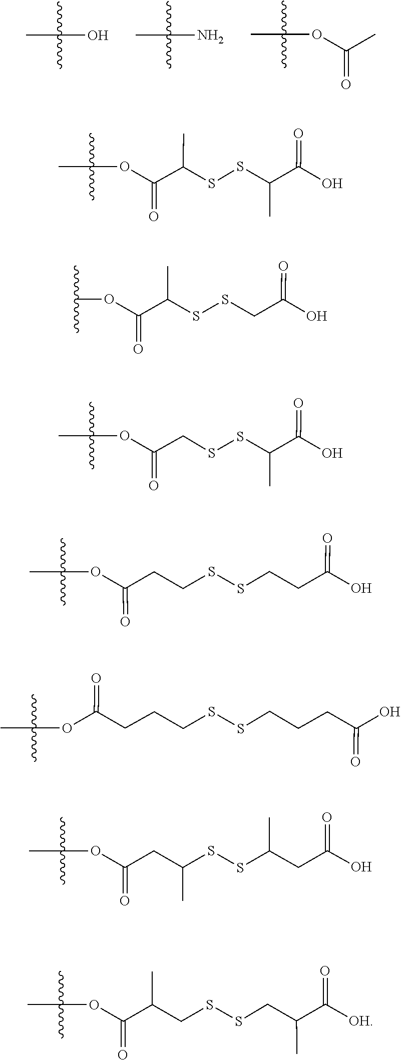

12. The compound according to claim 1, or a pharmaceutically acceptable salt or isomer thereof, wherein R₇ and R₇′ are independently selected from the group consisting of —CH₂—, —(CH₂)₂—, —CH₂CH(CH₃)—, —(CH₂)₃—, —CH₂NH—, and —(CH₂)₂NH—.

13. The compound according to claim 1, or a pharmaceutically acceptable salt or isomer thereof, wherein Rs is selected from the group consisting of —CH₂CH₃, —(CH₂)₂CH₃, —CH₂COOH, —(CH₂)₂COOH, —(CH₂)₃COOH, —CH₂CH(CH₃) COOH, and —CH(CH₃) CH₂COOH.

14. A compound of the following formulae, or a pharmaceutically acceptable salt or isomer thereof:

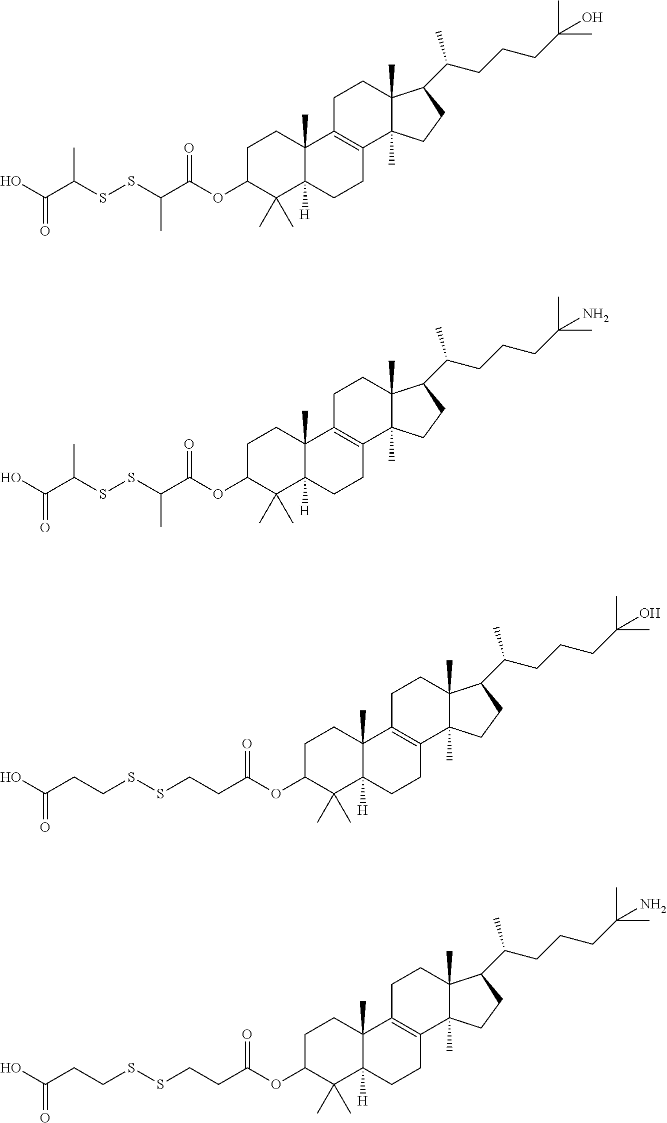 [SMILES: CC(SSC(C)C(=O)OC1CC[C@]2(C)C3=C(CC[C@H]2C1(C)C)[C@]1(C)CC[C@H]([C@H](C)CCCC(C)(C)O)[C@@]1(C)CC3)C(=O)O ; CC(SSC(C)C(=O)OC1CC[C@]2(C)C3=C(CC[C@H]2C1(C)C)[C@]1(C)CC[C@H]([C@H](C)CCCC(C)(C)N)[C@@]1(C)CC3)C(=O)O ; C[C@H](CCCC(C)(C)O)[C@H]1CC[C@@]2(C)C3=C(CC[C@]12C)[C@@]1(C)CCC(OC(=O)CCSSCCC(=O)O)C(C)(C)[C@@H]1CC3 ; C[C@H](CCCC(C)(C)N)[C@H]1CC[C@@]2(C)C3=C(CC[C@]12C)[C@@]1(C)CCC(OC(=O)CCSSCCC(=O)O)C(C)(C)[C@@H]1CC3]

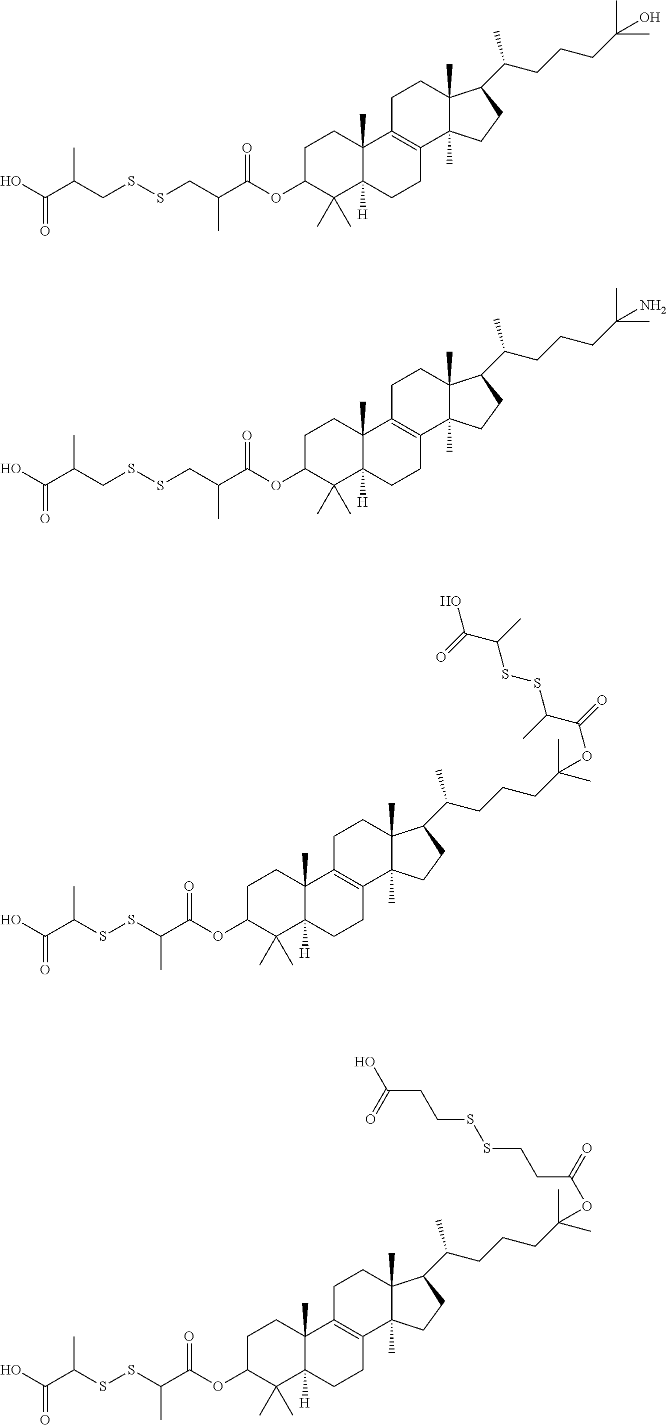 [SMILES: CC(CSSCC(C)C(=O)OC1CC[C@]2(C)C3=C(CC[C@H]2C1(C)C)[C@]1(C)CC[C@H]([C@H](C)CCCC(C)(C)O)[C@@]1(C)CC3)C(=O)O ; CC(CSSCC(C)C(=O)OC1CC[C@]2(C)C3=C(CC[C@H]2C1(C)C)[C@]1(C)CC[C@H]([C@H](C)CCCC(C)(C)N)[C@@]1(C)CC3)C(=O)O ; CC(SSC(C)C(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3)C(=O)O ; CC(SSC(C)C(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3)C(=O)O]

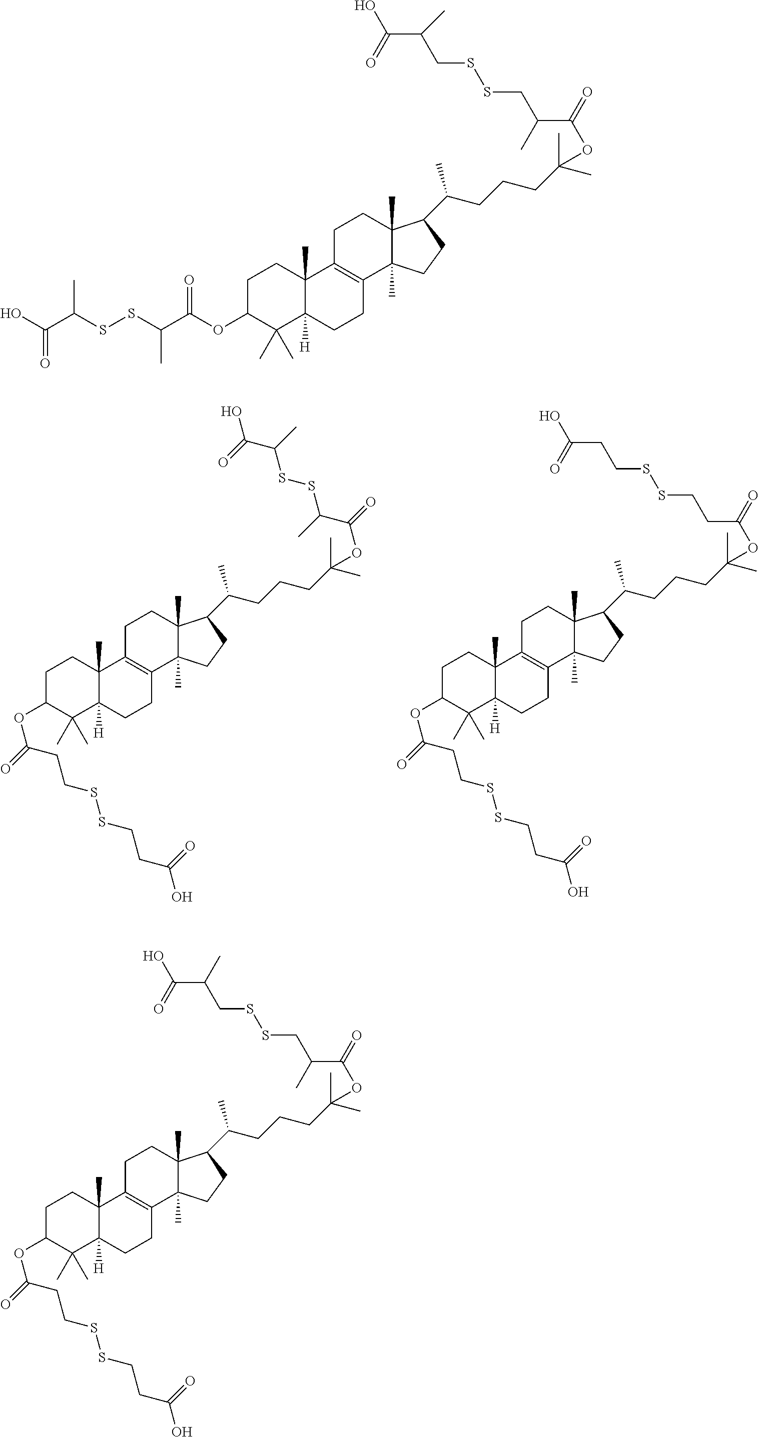 [SMILES: CC(CSSCC(C)C(=O)OC(C)(C)CCC[C@@H](C)[C@H]1CC[C@@]2(C)C3=C(CC[C@]12C)[C@@]1(C)CCC(OC(=O)C(C)SSC(C)C(=O)O)C(C)(C)[C@@H]1CC3)C(=O)O ; CC(SSC(C)C(=O)OC(C)(C)CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3)C(=O)O ; C[C@H](CCCC(C)(C)OC(=O)CCSSCCC(=O)O)[C@H]1CC[C@@]2(C)C3=C(CC[C@]12C)[C@@]1(C)CCC(OC(=O)CCSSCCC(=O)O)C(C)(C)[C@@H]1CC3 ; CC(CSSCC(C)C(=O)OC(C)(C)CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3)C(=O)O]

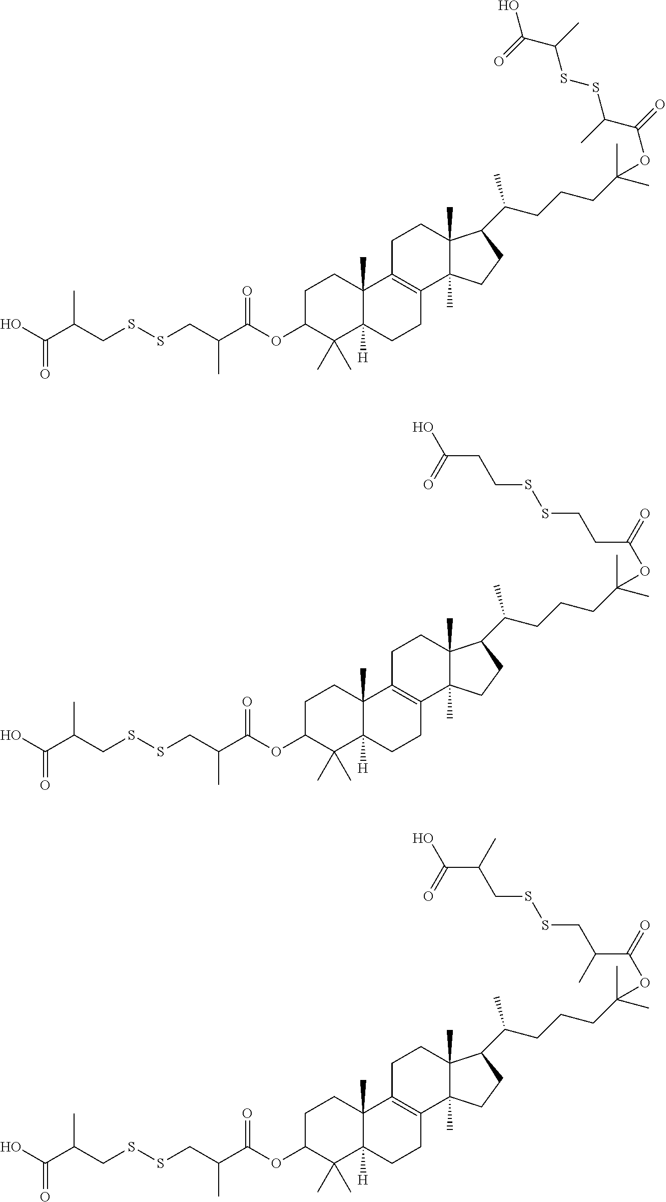 [SMILES: CC(CSSCC(C)C(=O)OC1CC[C@]2(C)C3=C(CC[C@H]2C1(C)C)[C@]1(C)CC[C@H]([C@H](C)CCCC(C)(C)OC(=O)C(C)SSC(C)C(=O)O)[C@@]1(C)CC3)C(=O)O ; CC(CSSCC(C)C(=O)OC1CC[C@]2(C)C3=C(CC[C@H]2C1(C)C)[C@]1(C)CC[C@H]([C@H](C)CCCC(C)(C)OC(=O)CCSSCCC(=O)O)[C@@]1(C)CC3)C(=O)O ; CC(CSSCC(C)C(=O)OC1CC[C@]2(C)C3=C(CC[C@H]2C1(C)C)[C@]1(C)CC[C@H]([C@H](C)CCCC(C)(C)OC(=O)C(C)CSSCC(C)C(=O)O)[C@@]1(C)CC3)C(=O)O]

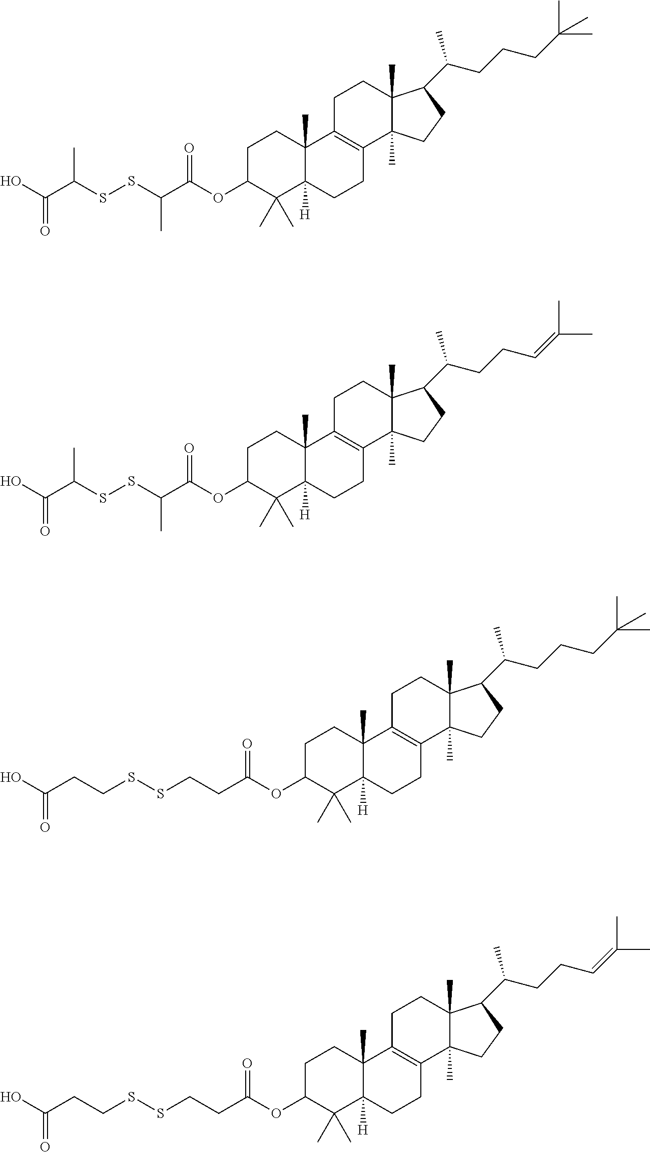 [SMILES: CC(SSC(C)C(=O)OC1CC[C@]2(C)C3=C(CC[C@H]2C1(C)C)[C@]1(C)CC[C@H]([C@H](C)CCCC(C)(C)C)[C@@]1(C)CC3)C(=O)O ; CC(C)=CCC[C@@H](C)[C@H]1CC[C@@]2(C)C3=C(CC[C@]12C)[C@@]1(C)CCC(OC(=O)C(C)SSC(C)C(=O)O)C(C)(C)[C@@H]1CC3 ; C[C@H](CCCC(C)(C)C)[C@H]1CC[C@@]2(C)C3=C(CC[C@]12C)[C@@]1(C)CCC(OC(=O)CCSSCCC(=O)O)C(C)(C)[C@@H]1CC3 ; CC(C)=CCC[C@@H](C)[C@H]1CC[C@@]2(C)C3=C(CC[C@]12C)[C@@]1(C)CCC(OC(=O)CCSSCCC(=O)O)C(C)(C)[C@@H]1CC3]

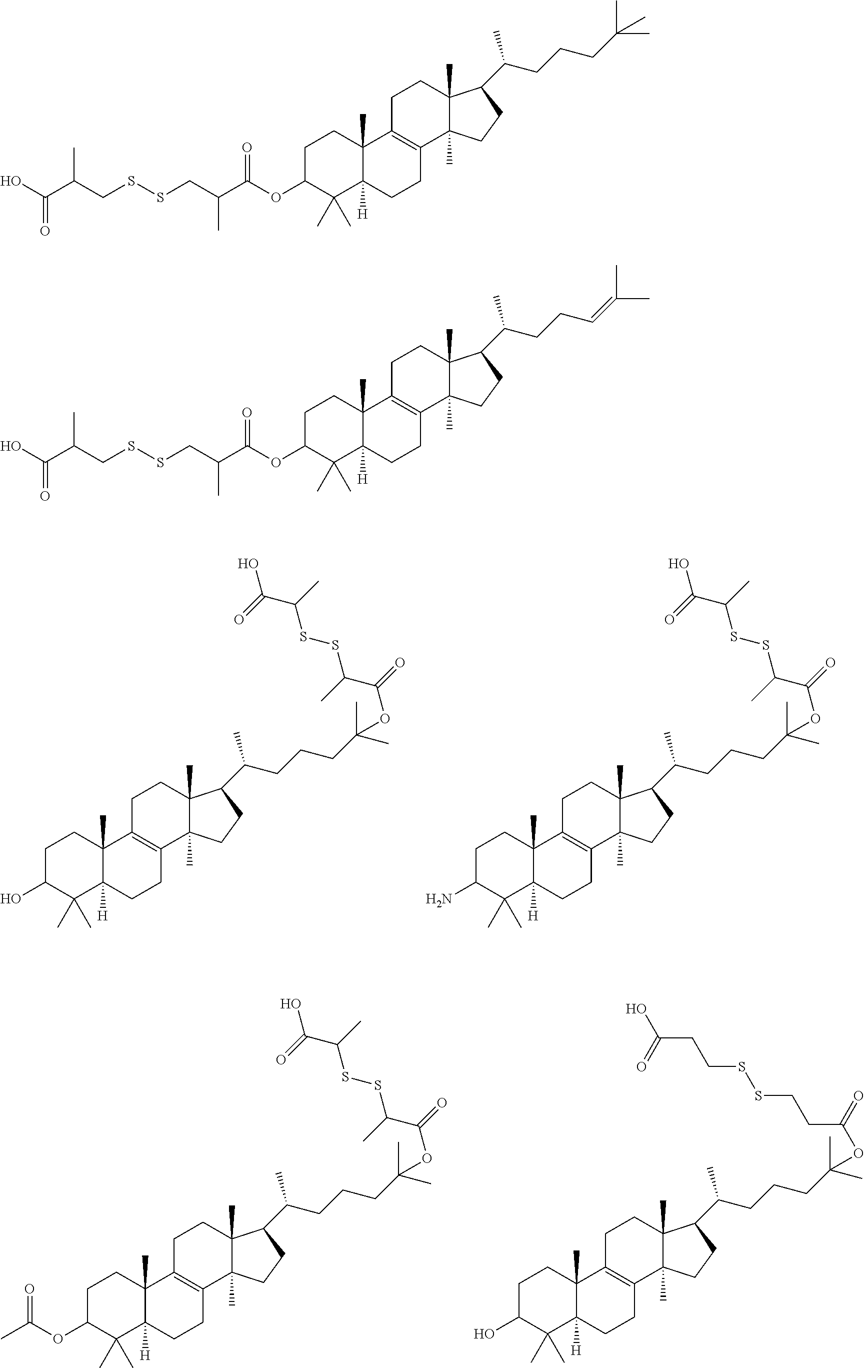

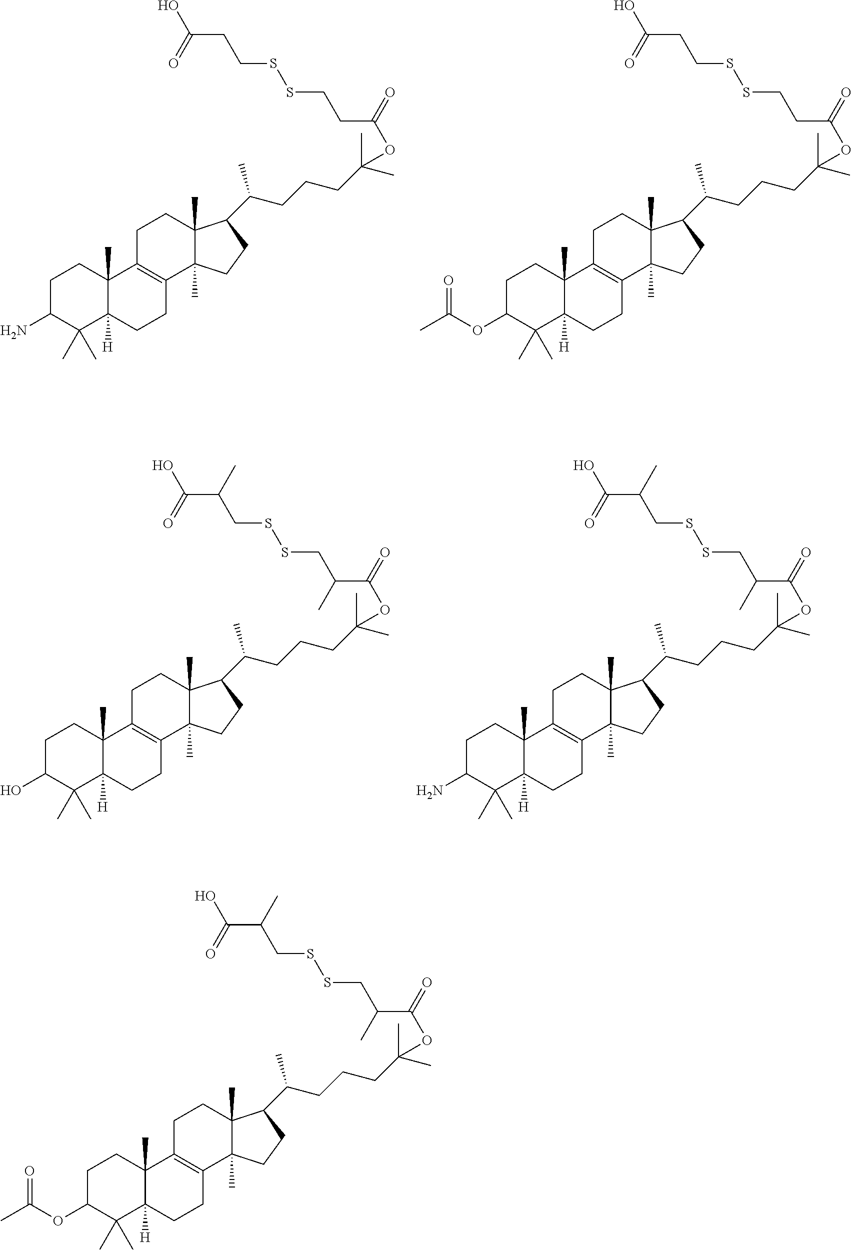

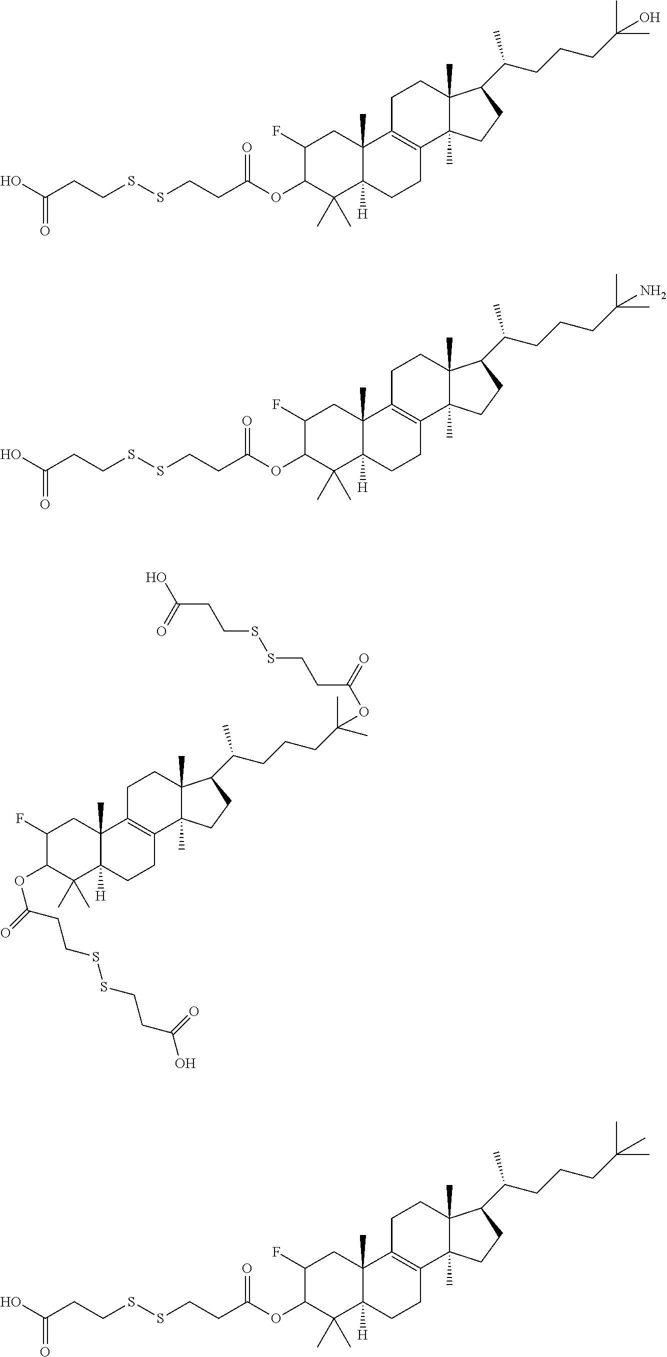 [SMILES: C[C@H](CCCC(C)(C)O)[C@H]1CC[C@@]2(C)C3=C(CC[C@]12C)[C@@]1(C)CC(F)C(OC(=O)CCSSCCC(=O)O)C(C)(C)[C@@H]1CC3 ; C[C@H](CCCC(C)(C)N)[C@H]1CC[C@@]2(C)C3=C(CC[C@]12C)[C@@]1(C)CC(F)C(OC(=O)CCSSCCC(=O)O)C(C)(C)[C@@H]1CC3 ; C[C@H](CCCC(C)(C)OC(=O)CCSSCCC(=O)O)[C@H]1CC[C@@]2(C)C3=C(CC[C@]12C)[C@@]1(C)CC(F)C(OC(=O)CCSSCCC(=O)O)C(C)(C)[C@@H]1CC3 ; C[C@H](CCCC(C)(C)C)[C@H]1CC[C@@]2(C)C3=C(CC[C@]12C)[C@@]1(C)CC(F)C(OC(=O)CCSSCCC(=O)O)C(C)(C)[C@@H]1CC3]

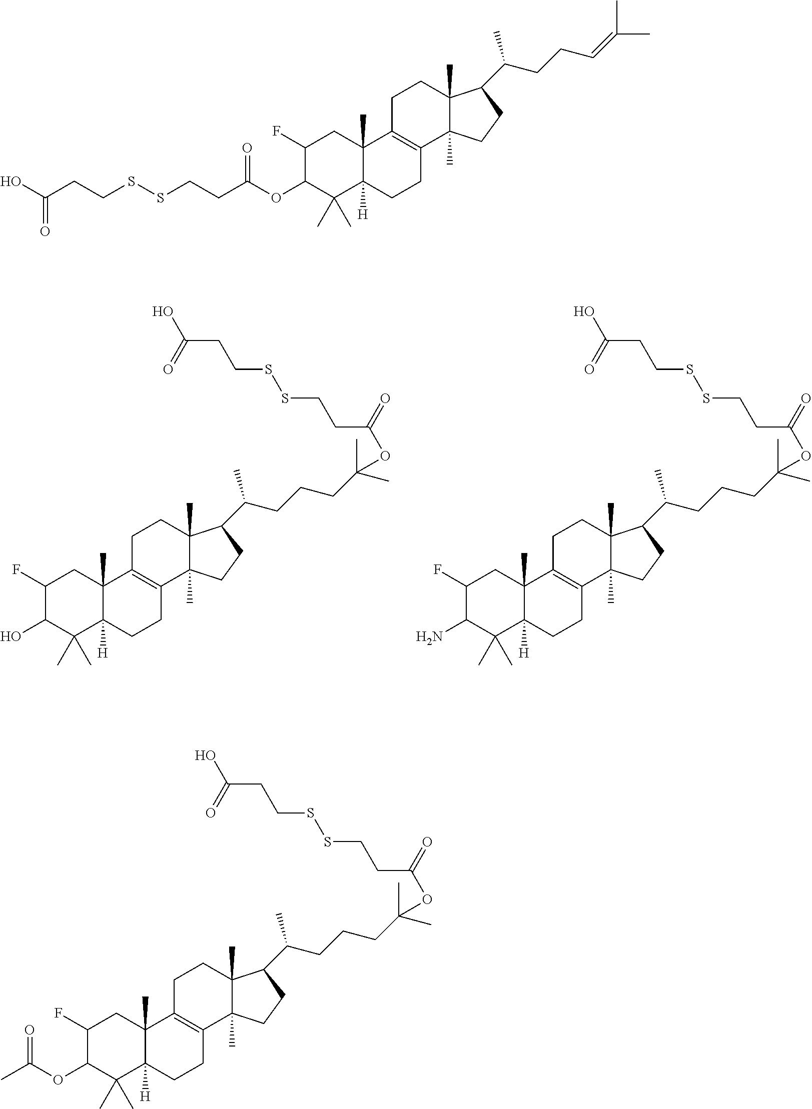

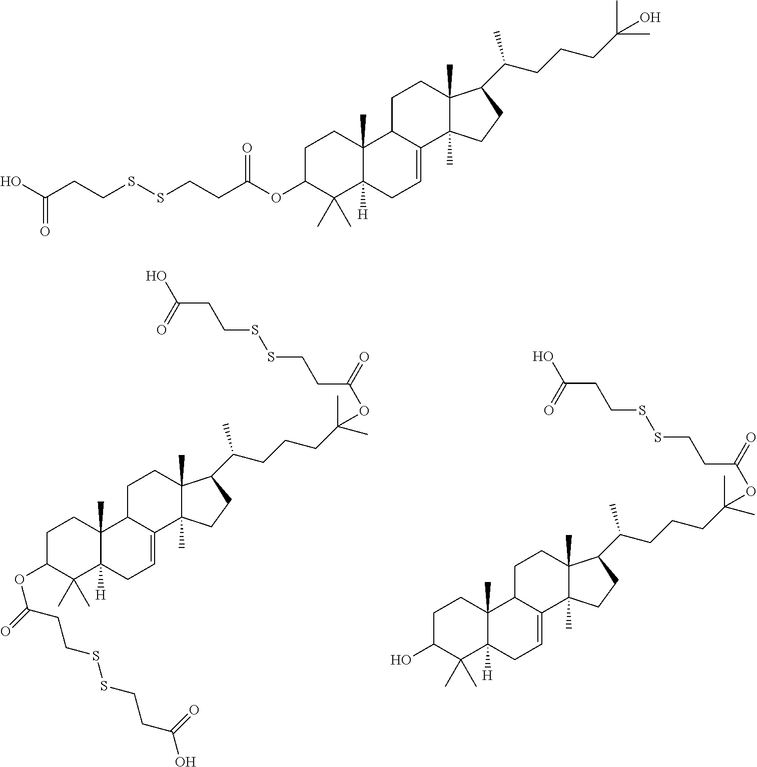 [SMILES: C[C@H](CCCC(C)(C)O)[C@H]1CC[C@@]2(C)C3=CC[C@H]4C(C)(C)C(OC(=O)CCSSCCC(=O)O)CC[C@]4(C)C3CC[C@]12C ; C[C@H](CCCC(C)(C)OC(=O)CCSSCCC(=O)O)[C@H]1CC[C@@]2(C)C3=CC[C@H]4C(C)(C)C(OC(=O)CCSSCCC(=O)O)CC[C@]4(C)C3CC[C@]12C ; C[C@H](CCCC(C)(C)OC(=O)CCSSCCC(=O)O)[C@H]1CC[C@@]2(C)C3=CC[C@H]4C(C)(C)C(O)CC[C@]4(C)C3CC[C@]12C]

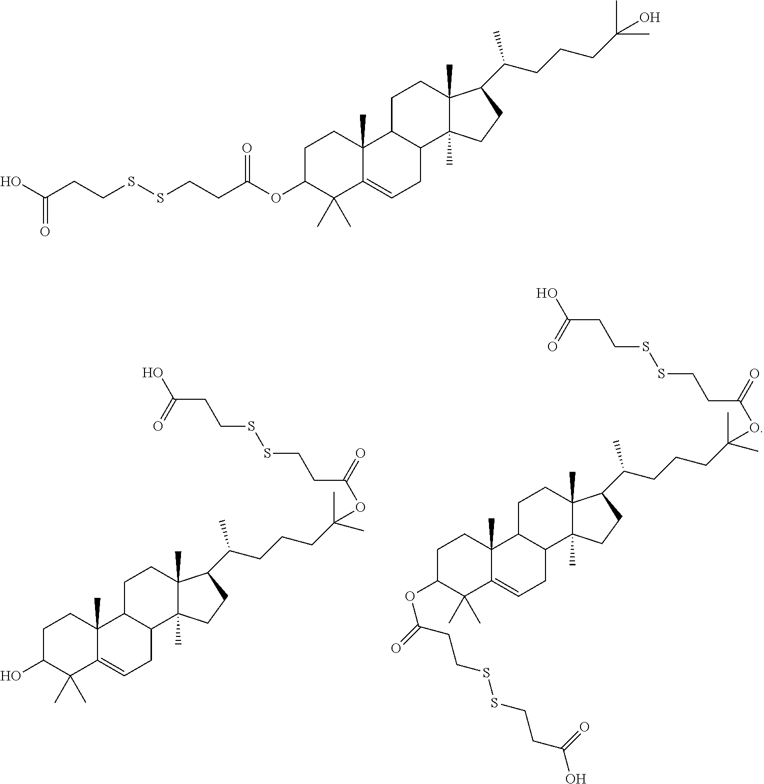 [SMILES: C[C@H](CCCC(C)(C)O)[C@H]1CC[C@@]2(C)C3CC=C4C(C)(C)C(OC(=O)CCSSCCC(=O)O)CC[C@]4(C)C3CC[C@]12C ; C[C@H](CCCC(C)(C)OC(=O)CCSSCCC(=O)O)[C@H]1CC[C@@]2(C)C3CC=C4C(C)(C)C(O)CC[C@]4(C)C3CC[C@]12C ; C[C@H](CCCC(C)(C)OC(=O)CCSSCCC(=O)O)[C@H]1CC[C@@]2(C)C3CC=C4C(C)(C)C(OC(=O)CCSSCCC(=O)O)CC[C@]4(C)C3CC[C@]12C]

15. A pharmaceutical composition comprising a therapeutically effective amount of the compound or the pharmaceutically acceptable salt or isomer thereof according to claim 1.

16. A method for treating an ophthalmic disease, comprising a step of administering a therapeutically effective amount of the compound or the pharmaceutically acceptable salt or isomer thereof according to claim 1, to a subject.

17. The method according to claim 16, wherein the ophthalmic disease for includes cataracts and/or presbyopia.

18. The method according to claim 17, wherein the compound, or the pharmaceutically acceptable salt or isomer thereof is administered via the eyes.

19. A method for preparing the compound or the pharmaceutically acceptable salt or isomer thereof according to claim 1, wherein the method is at least one selected from the following schemes: 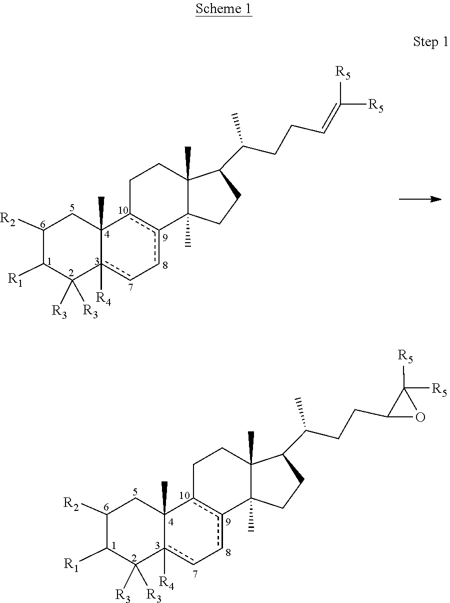 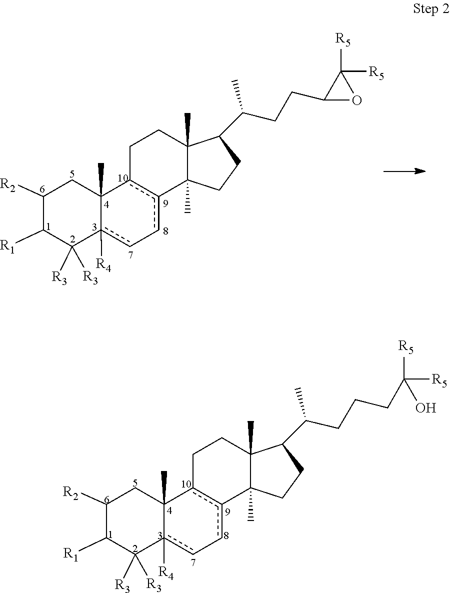 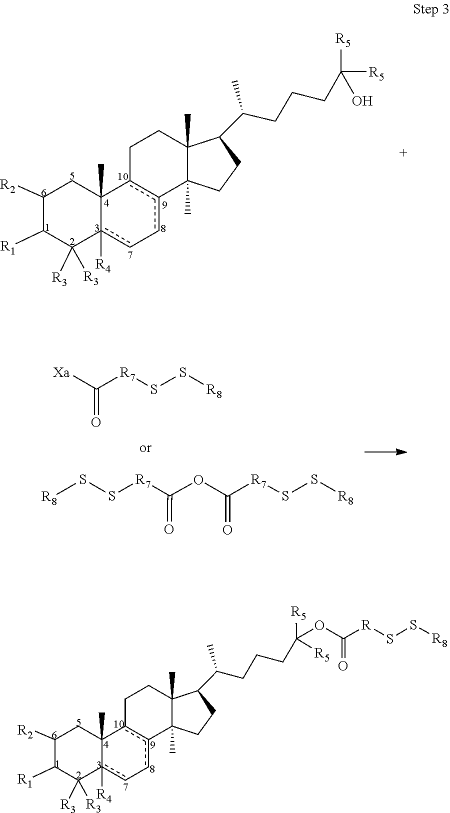 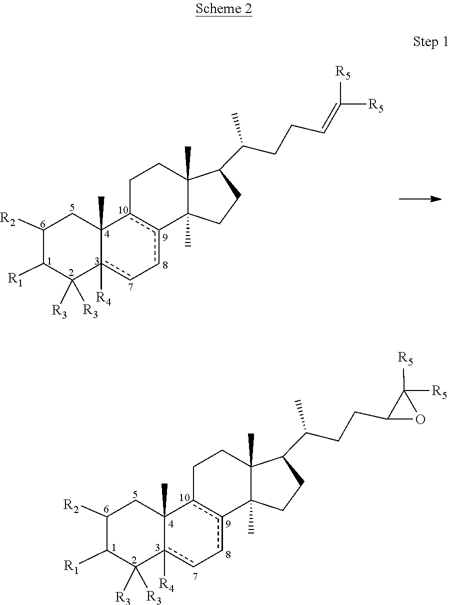 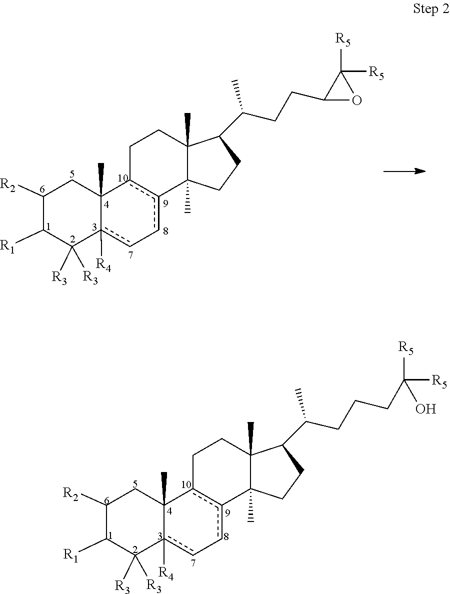 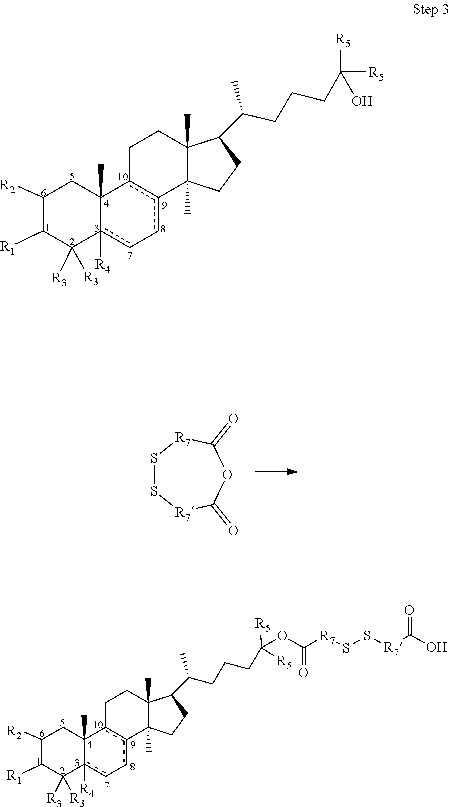 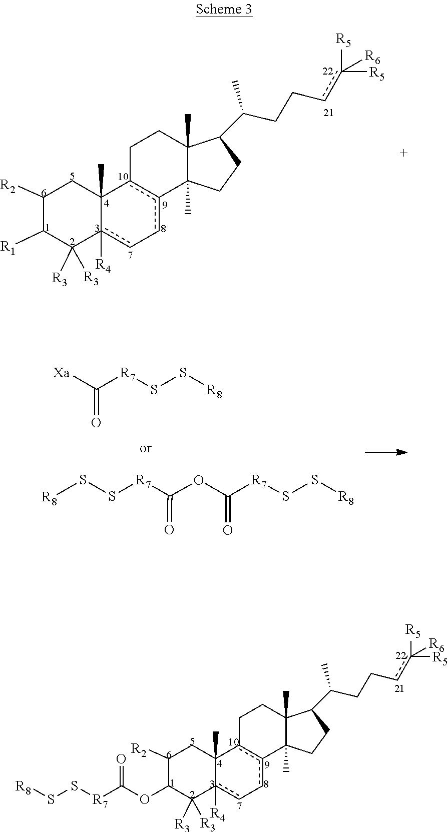 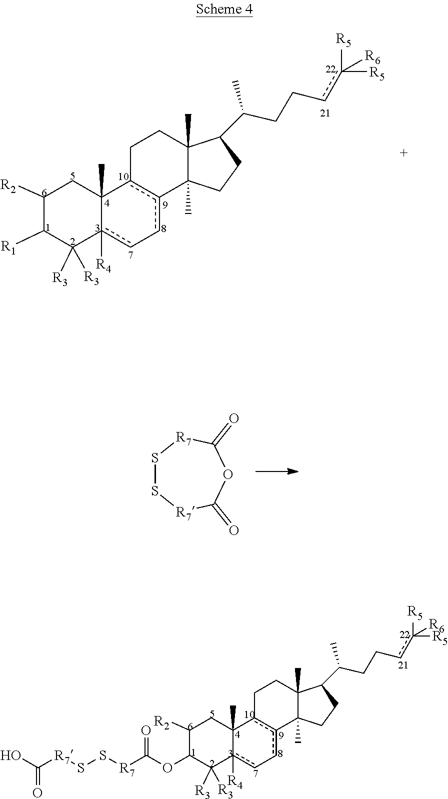
wherein, Xa represents a leaving group, for example, it is selected from the group consisting of hydroxyl and chlorine.
